(19) United States
(12) Patent Application Publication
(10) Pub. No.: US 2011/0150839 A1
(43) Pub. Date: Jun. 23, 2011

(54) METHOD TO OBTAIN TRANSGENIC PLANTS RESISTANT TO PHYTOPATHOGEN ATTACK BASED ON RNA INTERFERENCE (RNA)

(75) Inventors: Stefania Arciello, San Giorgio a Cremano (Napoli) (IT); Davide Andrenacci, Napoli (IT); Fabio Napoli, Napoli (IT); Maria Gabriella Colucci, Pozzuoli (Napoli) (IT)

(73) Assignee: ARTERRA BIOSCIENCE S.R.L., Napoli (IT)

(21) Appl. No.: 12/737,480

(22) PCT Filed: Jul. 15, 2009
(86) PCT No.: PCT/EP2009/005203

§ 371 Date: Mar. 4, 2011

(30) Foreign Application Priority Data
Jul. 17, 2008 (IT) MI2008A001305

(51) Int. Cl. A01N 63/00; A01H 1/00; A01H 5/00; A01P 1/00

(52) U.S. Cl. 424/93.2; 800/279; 800/301

(57) ABSTRACT

The present invention relates to a method to obtain transgenic plants resistant to the attack of phytopathogens (i.e. parasites and phytophages) based on RNA interference, which contemplates the expression of double strand RNA (dsRNA) in the plant tissues, suitable for inhibiting the functionality of a GPCR receptor, whose functioning is vital for fungi, herbivorous insects or phytopathogenic nematodes.

The present invention relates to a method to obtain transgenic plants resistant to the attack of phytopathogens based on RNA interference (RNAi) for the protection of the same from the attack of parasites and phytophages, which contemplates the expression of dsRNA in the plant tissues suitable for inhibiting the functionality of a GPCR receptor, whose functioning is vital for fungi, herbivorous insects or phytopathogenic nematodes. In particular, the present invention relates to plants expressing dsRNA in their tissues, obtainable with said preventive protection treatment from the attacks of fungi, herbivorous insects or phytopathogenic nematodes.

RNAi is a natural process preserved during evolution and present in all organisms. It is a gene silencing mechanism whereby various double-stranded RNA fragments are capable of interfering and extinguishing the gene expression. Once a double-stranded RNA molecule (dsRNA) has been defined, the so-called RNAi machinery is capable of activating the RNAi mechanism (Brandt, 2002).

Through an enzyme called dicer, the dsRNA sequence is cut into fragments having a shorter length (19-21 pair of bases) (Hamilton and Baulcombe, 1999). The short dsRNA fragment (called short interfering RNA, or siRNA) is associated with an enzymatic complex called RISC (RNA-interference silencing complex). The dsRNA is opened, probably by means of a helicase: only the antisense RNA strand remains associated with the RISC, whereas the sense strand is degraded. The RISC complex is capable of recognizing among the numerous mRNAs present in the cytosol, which is complementary to the antisense RNA fragment associated with the same complex. If the pairing between siRNA and mRNA is perfect (or almost perfect), a RISC component (called argonaute protein) is capable of effecting a cut on the mRNA (Hammond et al., 2001). The two resulting mRNA fragments, one without a head at 5′ and the other without a tail of A poles at 3′, are therefore rapidly degraded by the RNAse of the cell itself. Another common protein, although not universally present in the RNAi machinery, is RNA dependent RNA polymerase (RdRP) which synthesizes dsRNA starting from a single helix RNA mould to produce the RNAi amplification mechanism.

The discovery of RNAi as a powerful and easy gene silencing method has awakened the attention of the whole scientific community. The ubiquity of the phenomenon has allowed it to be studied on numerous species. Many experiments are in fact based on the simple immersion of entire organisms in solutions containing dsRNA or through feeding with bacteria expressing dsRNA. This has enabled the rapid identification of genes involved in the RNAi in C. elegans, and discovery of their homologues in Drosophila, plants and fungi, and has demonstrated that phenomena, which were first classified as quelling PTGS (post-transcriptional gene silencing), are all part of a single process whose roots are founded in a single ancestral mechanism.

In the plant context, applications of gene silencing by means of RNAi have allowed varieties of agricultural interest to be produced with increased resistance levels to diseases, insects, or with high nutritional quality.

Some researchers, for example, have contributed to improve rice plants using the RNAi technique. They have reduced the levels of glutenin producing a variety of rice called LGC-1 (low glutenin content 1) suitable for patients with renal deficiencies incapable of digesting glutenin (Kusaba et al., 2003).

Other RNAi applications concerned the removal of plant endotoxins by the silencing of genes involved in the biosynthesis of the toxin. The theobromine synthase enzyme of coffee plants was inactivated with dsRNA allowing the production of decaffeinated coffee (Ogita et al., 2003).

Silencing experiments using dsRNAs have also been carried out on pathogens and phytophages of plants of agricultural interest to inhibit the expression of some of their vital genes. Micro-injections of dsRNA in end phase larvae and adult coleopterans (Tribolium castaneum) have allowed genes to be silenced and their function to be studied (Tomoyasu and Denell, 2004). Some researchers have carried out tests with larvae of Diabrotica virgifera virgifera LeConte fed with a synthetic diet enriched with specific dsRNA to identify genes essential for the vitality of the insect. These tests led to the identification of 14 key genes whose inactivation due to interference causes larval death. Transgenic corn plants expressing one of these dsRNAs have shown high protection levels, comparable to those obtained with transgenic plants expressing the toxin Bt (Baum et al., 2007). Unlike the latter however, transgenic plants expressing dsRNA have a reduced environmental impact. The inhibition effect of insect genes, in fact, in this case depend exclusively on the specific recognition between the siRNA and target sequence to be inactivated. Problems linked to the introduction of “foreign” molecules into the environment and interactions with non-target organisms are therefore eliminated.

For the control of pathogen agents and phytophage organisms the selection of the gene target to be inactivated is of fundamental importance.

G protein coupled receptors (GPCRs) comprise a vast group of proteins, structurally and functionally similar to each other, which exert functions of vital importance in all eukaryotic organisms. They consist of a single polypeptide chain which crosses the membrane seven times with the extracellular amino-terminal end and the intracellular carboxy-terminal end; following the binding with extracellular ligands (peptides, small molecules, ions, light and aromatic compounds) the receptor is activated by triggering a response in the cell which leads to the production of a secondary message (cAMP, Ca²⁺, cGMP, IP₃).

Most organisms of agricultural interest have numerous GPCRs, the majority of which are fundamental for the correct exertion of their vital functions or for their pathogenicity. The most widely-studied species, whose genome is completely sequenced and registered and in which there is considerable information on the developmental biology, are obviously the model organisms: Drosophila melanogaster, Caenorhabditis elegans, Saccharomyces e Neurospora. D. melanogaster is the most widely-studied arthropod and is considered the model organism for the study of harmful insects. In Drosophila genome about 270 GPCRs have been identified, grouped into 5 families. The genome sequence of the mosquito Anopheles gambiae has also recently been completed. A detailed bio-informatic approach has led to the identification of 276 GPCRs in the genome of this dipteran of great sanitary interest. A great deal of information is also available on the expression specificity of these genes in the different development phases and a detailed comparative analysis has been effected with the genome of Drosophila melanogaster.

C. elegans is the model metazoan widely studied from a genetic, neurobiological, cellular and molecular point of view. Also for this organism the genome sequence is complete and a great deal of information concerning bio-informatics, expression, in addition to signal transduction, neurobiology, development, behaviour, reproduction is provided in literature. Projects financed by public institutions are also in an advanced phase, which are aimed at the identification of abut 315.000 ESTs (Expressed Sequence Tags) from 20 different species of nematode parasites. The availability of all this information will allow to clone the cDNAs of the GPCRs very rapidly and thus use them as targets of RNAis and potential nematocides.

Less information, on the other hand, is available for the GPCRs of fungi. A total of two transduction pathways mediated by GPCRs have been identified in both ascomycetes and basidiomycetes:

a) pheromone response (GPCRs=STE);

b) response to nutritional factors (glucose sensors).

Much more information is available on the heterotrimeric G proteins, direct effectors of GPCRs, in both ascomycetes and basidiomycetes (Li et al, 2007). At least 3 Gα subunits have been identified and null-mutants of various fungus species have been obtained (Saccharomyces, Neurospora, Aspergillus, Ustilago, Fusarium, Colletotrichus, Cryphonectria, etc.) which suggested the involvement of G proteins in a wide range of signaling pathways: pheromone response, sensitivity to nutrients, sterility, life growth, virulence and pathogenicity, development of the ascospores, morphogenesis, light. It is therefore evident that inactivating these GPCRs interferes with signals of vital importance of pathogen fungi.

In conclusion, interference by means of RNAi on the GPCRs of any organism would therefore be extremely specific as the percentage of gene homology among the GPCRs of insects, nematodes, fungi and mammals never exceeds 20-30% (see Table 1 below). Similarly, the percentage relating to the homology among the GPCRs of species belonging to the same order is never complete (around 90%) and this ensures discrimination between species of harmful organisms and those benevolent to the environment (data not published).

TABLE 1
Example of homology degrees between a given insect
species and other species of organisms:

human being ≅ 20-30%
insect belonging to the same order ≅ 90%
insect belonging to a different order ≅ 30-55%.

So far, most of the substances used as pesticides, nematocides or fungicides are molecules with a wide spectrum, there is therefore an enormous request for more selective methods which specifically interfere with the survival and fertility of harmful organisms, but at the same time have a minimum or no effect on non-target organisms and on the environment. The nematocides currently present on the market, for example, are highly toxic, costly and difficult to use. Methylene bromide which is the most widely-used product for the control of nematodes, fungi and bacteria on fruit trees and vegetables in California and Florida, is a neuro-toxin which also reduces the ozone in the atmosphere. Other nematocides used are aldicarb (Temik) and 1′1,3-dichloropropene (TeloneII), both carcinogenic for human beings.

On the basis of what is specified above, there is an evident demand for new methods and relative biological targets which specifically interfere with the survival and fertility of phytopathogen organisms (i.e. parasites and phytophages) more selectively and consequently having a lower environmental impact.

The Applicant has now found that GPCR receptors can be convenient targets for the control of parasites and phytophages of plants, such as fungi, insects and nematodes. Many of these GPCRs, in fact, have a gene sequence which is very specific for harmful organisms, consequently molecules of dsRNA are capable of inhibiting their functionality and cannot damage the host organism (plant) or a non-target consumer of the same plant (i.e. human being, other animals). In particular, the Applicants have prepared a method which uses the expression of dsRNA for inhibiting the functionality of a GPCR receptor, whose functioning is vital for fungi, herbivorous insects or phytopathogen nematodes.

The advantages which can be associated with the technology, object of the present invention, are summarized hereunder:
a) the selectivity, which can harm a herbivorous insect, nematode or parasitic fungus but not other benevolent organisms (i.e. bees);
b) food safety, for the same reason as above, but also for the fact that an RNA molecule can be easily degraded and does not persist in the environment;
c) the possibility of expressing dsRNA also in portions of the plant which would normally be particularly difficult to reach even with systemic chemical treatment (e.g. pyramid);
d) the potential absence of allergenic risk, as new proteins of the host plant are not expressed;
e) the possibility of selecting plants with a high/total proteomic homology with wild-type with a suitable selection of clones.

The Applicant has now found a method for obtaining transgenic plants resistant to the attack of one or more phytopathogens by RNA interference comprising the following phases:
a) isolation of the cDNA nucleotide sequence encoding for a G protein coupled receptor (GPCR), or a portion thereof, whose function is vital for the phytopathogen;
b) construction of an expression vector comprising the cDNA nucleotide sequence encoding for a GPCR or a portion thereof as determined by step a), flanked by two specific recombination sites;
c) recombination reaction of the expression vector of step b) with a binary vector comprising a constitutive or tissue-specific promoter, the same specific recombination sites and an intronic sequence to obtain a recombinant plasmid;
d) transfer of said recombinant plasmid into competent cells of Agrobacterium tumefaciens and transformation of the plant;
e) growing the plant and obtaining the constitutive or tissue-specific expression of dsRNA in said plant.

In particular, this method uses the double-stranded RNA expression for inhibiting the functionality of a GPCR receptor, whose functioning is vital for fungi, herbivorous insects or phytopathogen nematodes, and said dsRNA is expressed in the plant tissues so that it can be absorbed through the fungal haustorium or ingested by the insect or nematode.

Said phytopathogens are preferably selected from insects of the lepidopteran or coleopteran type often herbivorous when in the larval state, nematodes and fungi. According to preferred embodiments, the herbivorous insect in the adult or larval stage can be Spodoptera littoralis, Heliotis virescens. According to further preferred embodiments, said nematode is selected from Meloidogyne incognita, Meloidogyne hapla, Globodera rostochiensis. Again, according to a preferred embodiment, phytopathogen fungi can be selected from oidium, rust, septoriae, venturia, alternaria. These fungi can be diffused in the phylloplane, or in the hypogean parts of the plant, and settle in the surface portions, as also in the more internal tissues.

In the spirit of the invention, one or more dsRNA can be constitutively expressed in the tissues of the whole plant, or in certain portions, such as the phylloplane, or in the root system, using specific promoters (tissue-specific promoters). The expression in the hypogean parts of the plant can be aimed at controlling, for example, terrestrial phytopathogens, whereas that in the root system used for the control of nematodes or other harmful insects. Consequently, according to preferred embodiments of the invention, when the dsRNA expression is tissue-specific, said tissue is selected from the groups which consists of phylloplane, trunk, root system, buds, flowers, fruit.

The method according to the present invention envisages the selection of one or more GPCR receptors as RNA interference target, said receptor must be vital for the phytopathogen against which the plant is to be made resistant. In relation to the insect, nematode or fungus various types of GPCR receptor can be used as target. Said GPCR target receptor is preferably selected from the group which consists of allatostatin C or A, octopamin/tyramine, PBAN peptide, diuretic hormone, adipokinetic hormone, neuropeptides, dopamine, serotonin and rhodopsin-like receptors.

According to a preferred embodiment, in which, when said herbivorous insect is Spodoptera littoralis, the nucleotide sequence of the cDNA encoding a GPCR receptor or a portion thereof is selected from the group which consists of AlstAR (SEQ ID NO:1), AlstCR (SEQ ID NO:2), DHR (SEQ ID NO:3), AKHR (SEQ ID NO:4), LGR1 (SEQ ID NO:5), Oct/TyrR (SEQ ID NO:6), OR83b (SEQ ID NO:7) e PBAN-R (SEQ ID NO:8). With respect to other lepidopteran species, such as for example, Heliotis virescens, the authors have found that GPCR receptor homologous to that of allatostatin C of Spodoptera could represent the election target for this type of herbivorous insect.

With respect to the use of the method according to the invention for obtaining transgenic plants resistant to nematode parasites, in particular of the species Meloidogyne incognita, the authors of the present invention have found six possible GPCR target silencing receptors by means of RNAi. The nucleotide sequence of the cDNA encoding the above GPCR receptors is selected from the group which consists of serotonin-like receptors (homologue of the serotonin receptors of C. elegans, SEQ ID NO:9), dopamine-like receptor (homologue of the receptor of dopamine CeDop-1 of C. elegans, SEQ ID NO:10), neuropeptide receptor (homologue of the receptor of neuropeptides of C. elegans Q2TGX5_MELIC, SEQ ID NO:11) and rhodopsin-like receptors (homologues of GPCR of C. elegans (T27D1.3; T07D4.1; B0563.6; F02E8.2) belonging to the family of rhodopsin-like GPCRs.

According to a preferred embodiment of the method of the present invention, one or more dsRNA can be expressed in the tissues of the transgenic plant obtained. This way, it is possible to obtain lines expressing two or three different types of dsRNA and the same plants can therefore be resistant to the attack of both phytophages (which prevalently feed on leaves) and parasites of the roots. Although preserving the specificity of resistance to single species of harmful organisms, plants can be produced, which are resistant to various species, even very different from each other, i.e. belonging to phylogenetically completely distant groups (Phyla). Consequently, according to a particularly preferred embodiment of the invention, phases a) to c) of the method according to the invention can be repeated in parallel using in each phase, a nucleotide cDNA sequence encoding a GPCR receptor vital for different phytopathogens (i.e. nematode or insect, different species of nematodes, insects or fungi) to obtain one or more recombinant plasmids with which d) co-transforming the same plant or d1) transforming different plants and crossing them. Thanks to crossbreeds between transformed plants, plants which express specific dsRNAs both against insect GPCRs (i.e. Spodoptera, Heliotis), and against nematode GPCRs (M. incognita), can be created as well.

The present invention also envisages the use of the RNA interference technology for protecting plants of agronomical interest (such as, for example, cereals, solanaceous plants, vines, vegetables), ornamental plants (such as roses, etc.), environmental plants (such as conifers, linden trees, poplars, etc.). According to a preferred embodiment of the invention a solanaceous plant is used, preferably tobacco, more specifically Nicotiana tabacum.

A further object of the present invention relates to a transgenic plant resistant to one or more phytopathogens which can be obtained according to the method as defined above. Plants genetically modified for inhibiting GPCR receptors of phytopathogens, according to the procedures envisaged by the present invention, can be used for the production of food destined for human beings or for zootechnics, but also for the production of biomasses for industrial purposes (paper, fibres, pharmacological substances, biomasses for energy production or basic chemical substances, etc.). According to a preferred embodiment of the present invention, said transgenic plant is a tobacco plant resistant to one of its most harmful parasites, the insect Spodoptera littoralis. In particular, the transgenic tobacco plant (i.e. Nicotiana tabacum) expresses a dsRNA capable of inhibiting a GPCR receptor of S. littoralis (selected from AlstAR (SEQ ID NO:1), AlstCR (SEQ ID NO:2), DHR (SEQ ID NO:3), AKHR (SEQ ID NO:4), LGR1 (SEQ ID NO:5), Oct/TyrR (SEQ ID NO:6), OR83b (SEQ ID NO:7) and PBAN-R (AF401480, SEQ ID NO:8), through the ingestion of plant tissue expressing said dsRNA by the same insect in the larval stage. In a particularly preferred embodiment, the present invention relates to a transgenic plant resistant to Spodoptera littoralis characterized in that it is a tobacco plant and expresses dsRNA in which the antisense strand is complementary to the mRNA encoding the receptor AlstCR (SEQ ID NO:2) or DHR (SEQ ID NO:3).

According to another preferred embodiment the invention relates to a transgenic plant resistant to Meloidogyne incognita characterized in that it is a tobacco plant and expresses a dsRNA wherein the antisense strand is complementary to the mRNA encoding for SEQ ID NO:11.

An object of the present invention therefore relates to a method for the prophylactic treatment of plants to make them resistant to the attack of one or more insects, nematodes and phytopathogen fungi comprising phases a)-e) of the method defined above. In order to ensure their best development, the plants thus obtained can, if necessary, be subjected to a treatment phase with fertilizers, growth regulators or biostimulants, or with fungicidal products, herbicides, insecticides and nematocides, synthetic or natural. These applications could allow a lesser risk of selecting parasites resistant with respect to the biocide activity obtained with the technology object of this patent, and it is possible to have a synergic action between the application of a pesticide and the biocide activity obtained with the technology object of the present invention.

The present invention will now be described for illustrative but non-limiting purposes, according to its preferred embodiments with particular reference to the figures of the enclosed drawings, in which:

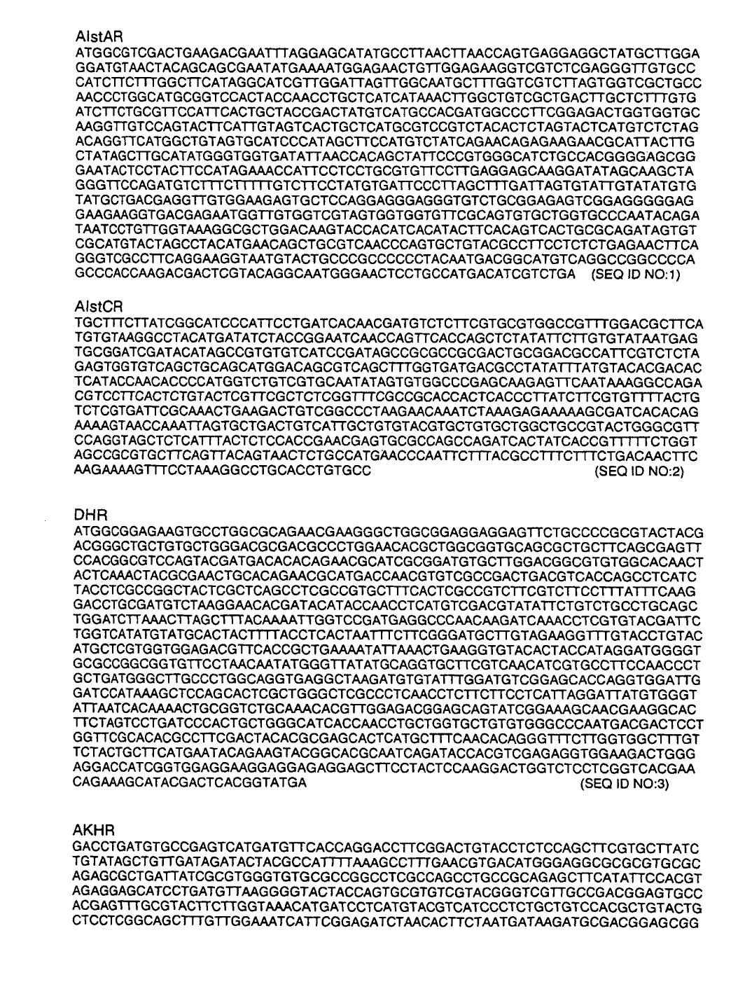

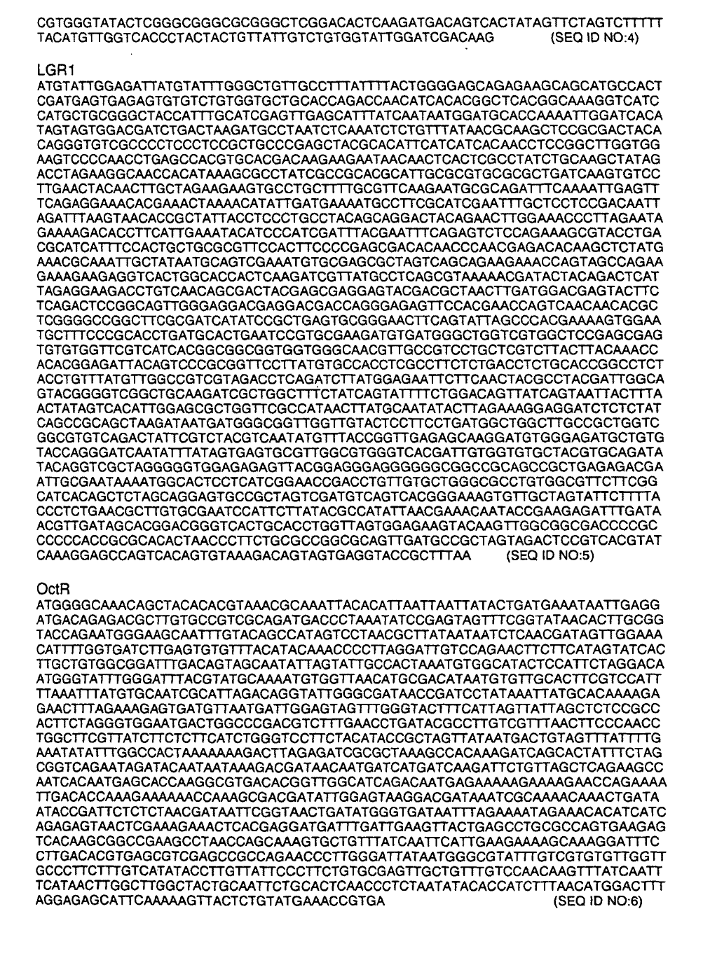

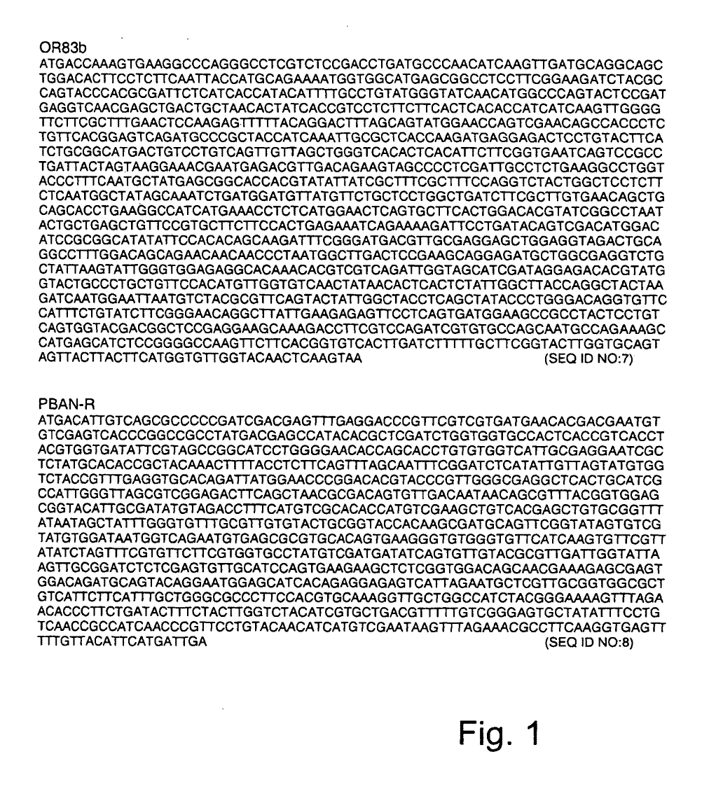

FIG. 1 shows the sequences of the cDNAs encoding the GPCR receptors: AlstAR, AlstCR, DHR, AKHR, LGR1, OctR, OR83b, PBANR cloned from S. littoralis;

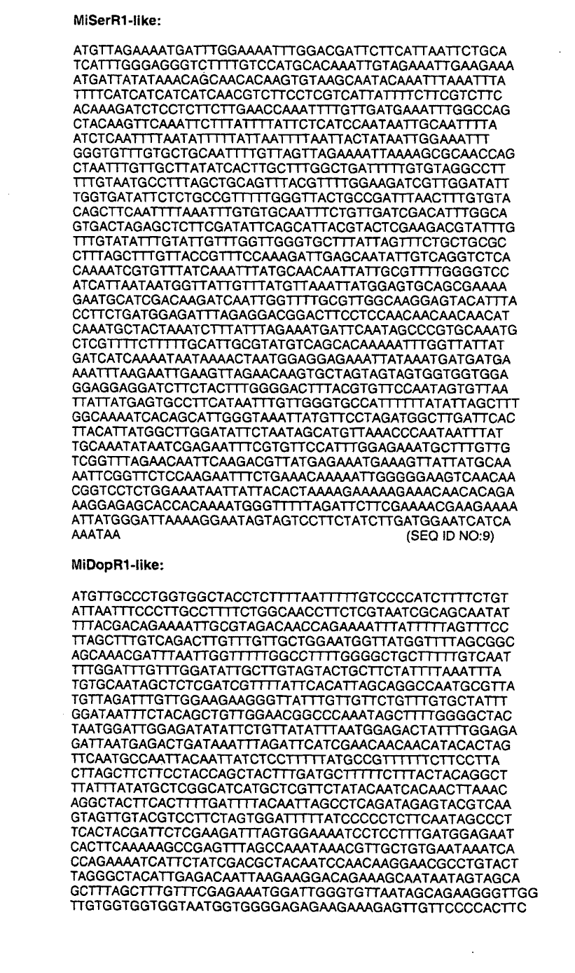

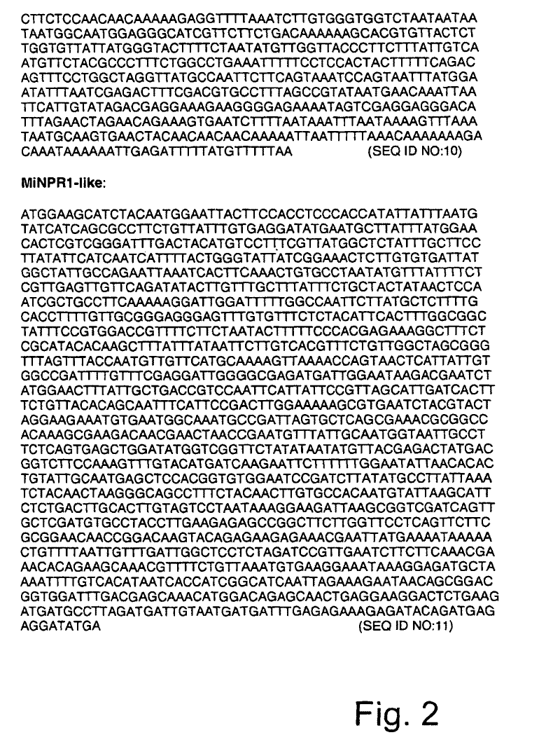

FIG. 2 shows the sequences of the cDNAs encoding the GPCR receptors: MiSerR1-like, MiDopR1-like and MiNpR1-like cloned from Meloidogyne incognita;

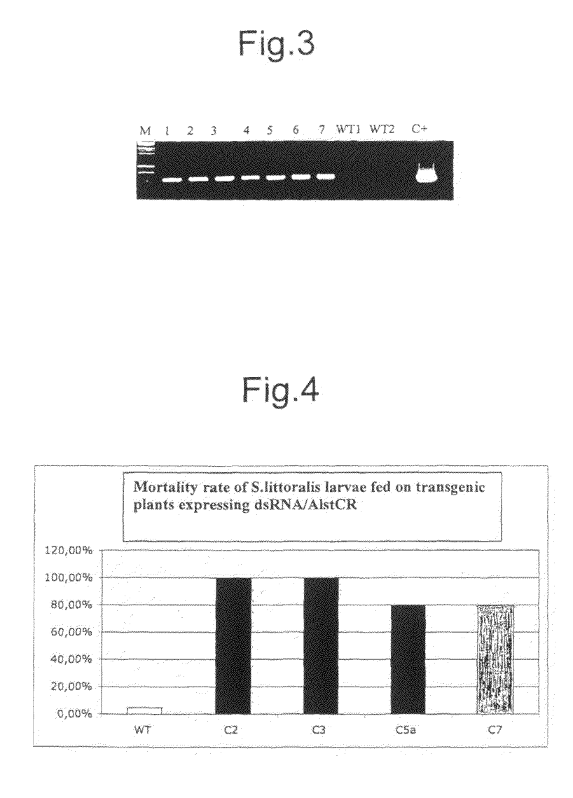

FIG. 3 shows the RT-PCR analysis of transgenic plants AlstCR (lanes 1 to 7); WT1 and WT2: non-transformed tobacco plants; M: known molecular weight marker; C+: plasmid pENTR+AlstCR;

FIG. 4 shows the histograms relating to the mortality rate after 4 weeks of Spodoptera littoralis larvae fed on transgenic tobacco plants expressing dsRNA specific for AlstCR;

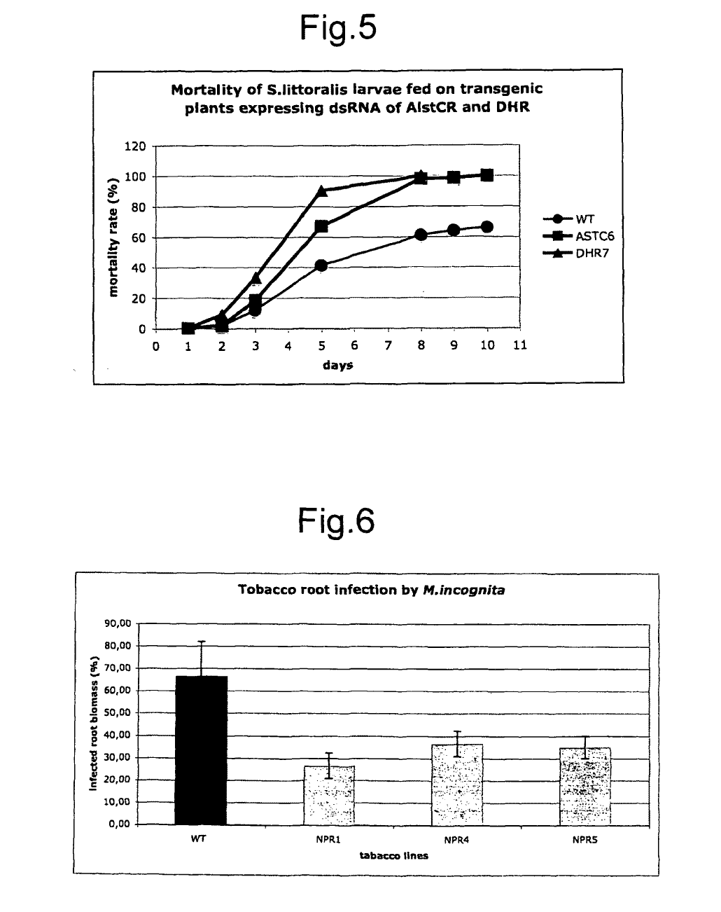

FIG. 5 shows the trend of the mortality rate of Spodoptera littoralis larvae fed on transgenic tobacco plants expressing dsRNA specific for AlstCR and DHR;

FIG. 6 shows the histograms relating to the percentage of infected tobacco roots by M. incognita of wild-type plants and transgenic plants expressing MiNpR1-like/dsRNA.

For illustrative but non-limiting purposes of the present invention, we describe two examples of transformations of a solanaceous plant, namely tobacco, to protect said plant from two of its most harmful parasites, the insect Spodoptera littoralis and the nematode Meloidogyne incognita.

### EXAMPLE 1

### Transformation of Tobacco Plants, in Order to Protect them from the Insect Spodoptera littoralis

Materials and Methods

Selection of GPCR Targets

The receptors of S. littoralis selected as targets of the present embodiment of the invention are:

1. AlstAR (putative receptor of allatostatin A);

2. AlstCR (putative receptor of allatostatin C):

these two GPCRs are of fundamental importance as they bind the allatostatins, neuropeptides whose main function is to inhibit the synthesis of the juvenile hormone on the part of the “corpora allata”. In addition to being involved in the metamorphosis activation process (Weaver et al., 1994), allatostatins are also important for regulating contractions of the intestinal smooth muscles and in the development and functionality of the ovaries (Aguilar et al., 2003; Meyering-Vos et al., 2006);

3. DHR (receptor of the diuretic hormone DH): important for diuresis regulation (Johnson et al., 2004);

4. PBAN-R (receptor of the activation neuropeptide of the biosynthesis of pheromones, PBAN): it binds a small peptide which promotes the synthesis and release of sexual pheromones (Rafaeli et al., 2007);

5. AKHR (putative receptor of the adipokinetic hormone AKH): important for the metabolism of carbohydrates and lipids and also for flight performances (Staubli et al, 2002);

6. LGR1 (Leucine-Rich Repeat-Containing G Protein-Coupled Receptor): belongs to the group of G protein-coupled receptors containing Leucine-rich repetitions (LGR) and exerts a fundamental role in reproduction (Nishi et al., 2000);

7. Oct/TyrR,putative receptor of octopamine and tyramine:

it is capable of interacting with neurotransmitters such as octopamine and tyramine and is therefore important in neuromodulation processes in sensorial and secretory organs (Farooqui, 2007);

8. OR83b (receptor of the family of olfactory receptors):

unlike other members of the family of olfactory receptors (OR), which are only expressed in small sub-populations of sensorial olfactory neurons, OR83b is expressed in almost all antenna neurons (Neuhaus et al., 2005). Rather than having a direct role in the olfactory function, it interacts with the conventional OR members and is essential for their localization from the cellular bodies to the sensory cilia where interaction with the odorant molecules takes place. For the general role it exerts in the olfactory system, OR83b is certainly of fundamental importance for the perception of odors and therefore for the nutrition of insects.

In order to perform RNAi experiments, all the sequences encoding GPCRs were cloned from Spodoptera with the exception of the gene encoding PBAN-R already present in the data bank (AF401480).

The sequences encoding the receptors AlstAR, DHR, LGR1, OR83b, Oct/TyrR, and part of the sequence encoding the receptor AlstCR and the receptor AKHR, were isolated using the cDNA back-transcribed by the total RNA extracted from S. littoralis, using degenerated primers deriving from the comparison of conserved regions among these genes in various insect species. For PBAN-R, on the other hand, specific primers for isolating the encoding sequence starting from V-phase larvae, were used. For the other receptors, on the basis of expression data available in literature, the sequences were isolated using different tissues and growth phases of the insect. In particular, the gene encoding the receptors LGR was amplified from eggs, whereas DHR from V-phase larvae; the gene encoding the receptors AlstAR, AlstCR, AKHR, OR83b, Oct/TyrR were amplified by RNA extracted from the head of individual adults. The PCR products were sequenced and used for designing specific primers for the 5′ and 3′ RACE-PCR experiments. The isolated sequences were cloned in the plasmid pCR® 2.1-TOP® (Invitogen) and entirely sequenced (FIG. 1).

Isolation and Cloning of the Selected Receptors

Cloning of the Receptor AlstAR

In order to isolate the cDNA encoding the receptor of Allatostatin A of Spodoptera littoralis, degenerated primers were designed on conserved regions determined by the alignment among the amino acid sequences of homologous receptors of Bombyx mori, Drosophila melanogaster e Periplaneta americana. The sequences of the primers are indicated below (the nucleotides in brackets indicate degenerated positions; the primer i.e. is a mixture of oligonucleotides, each containing a different nucleotide in that specific position):

1Fw:
(SEQ ID NO: 12)
atg(ac)g(acgt)(at)(gc)(acgt)ac(agct)ac(agct)aa
(tc)(tc)t(agct)(tc)t(agct)at(tc)a(ag)(tc)
2Fw:
(SEQ ID NO: 13)
gt(agct)cc(agct)tt(tc)ac(agct)gc(agct)ac(agct)ga
(tc)ta(tc)gt(agct)atg
1Rv:
(SEQ ID NO: 14)
gt(agct)ac(agct)(ac)g(agct)atggt(agct)gt(agct)gt
(agct)gt(agct)gt(agct)
2Rv:
(SEQ ID NO: 15)
tgg(at)s(agct)tg(tc)gt(agct)aa(tc)cc(atcg)gt
(atgc)(ac)t(tc)ta(tc)gc(agct).

The total RNA was extracted from heads of individual adults of Spodoptera littoralis using a kit commercialized by Promega. The samples of RNA were subjected to a treatment with rDNasi1 (Ambion) to eliminate the contaminating genomic DNA. 2 μl of RNA were charged onto 1% agarose gel in presence of denaturing loading dye and quantified using a specific marker for RNA (Fermentas) as reference. Gene tools software (Perkin Elmer) was used for the quantification. 1 μg of total RNA was transcribed into cDNA using the RevertAid M-MuLV Reverse Transcriptase enzyme (Fermentas) and 500 ng of oligodT (Promega).

A fragment of 507 by was obtained from the amplification of the cDNA through 30 PCR cycles (10 s at 98° C., 30 s at 50° C., 30 s at 72° C.) followed by 10 extension minutes at 72° C. The reaction was performed in a volume of 50 μl containing each primer at a concentration of 2.5 μM, 0.2 mM of deoxynucleotides (Fermentas) and 1 unit of Phusion High-Fidelity DNA Polymerase (Finnzymes) in the corresponding buffer.

The fragment obtained was cloned in the cloning vector pCR2.1 (Invitrogen) and subsequently sequenced (Ceinge Biotecnologie Avanzate scarl) using the oligonucleotides M13Fw and M13Rv, specific for the vector.

On the basis of the sequence, the following groups of oligonucleotides were designed for the 3′ RACE (Group 1; sense) and 5′ RACE (Group 2; antisense) respectively:

Group 1:

| | | |
|---|---|---|
| 1s | GCATCCCATAGCTTCCATGT | (SEQ ID NO: 16) |
| 2s | GGTGATATTAACCACAGCTATTCCCGTGGGC | (SEQ ID NO: 17) |
| 3s | GTATGCTGACGAGGTTGTGGAAGAGTGCTCC | (SEQ ID NO: 18) |
| 4s | AAGGTGACGAGAATGGTTGTG | (SEQ ID NO: 19) |
| 5s | GCGCAGATAGTGTCGCATGTA | (SEQ ID NO: 20) |

Group 2:

| | | |
|---|---|---|
| 1as | AGACGACTCTTCCACAACCTCGTCAGCATAC | (SEQ ID NO: 21) |
| 2as | GTTAATATCACCACCCATAT | (SEQ ID NO: 22) |
| 3as | GCCCACGGGAATAGCTGTGGTTAATATCACC. | (SEQ ID NO: 23) |

The 5′ and 3′ ends of the gene were obtained by means of PCR-RACE using the 5′/3′ RACEKIT in which the anchor primer and oligo(dT) anchor primer were substituted with oligo SL1 and oligo(dT)SL1 whose sequences are indicated hereunder:

SL1
(SEQ ID NO: 24)
GGTTTAATTACCCAACTTTGAG
dTSL1
(SEQ ID NO: 25)
GGTTTAATTACCCAACTTTGAGTTTTTTTTTTTTTTTT(ACG).

1 μl of cDNA, synthesized using 1.87 μM of the primer dTSL1, was amplified with the primers is and SL1 to obtain the 3′ end of the gene. The amplification conditions were:

40 cycles comprising 10 s at 98° C., 30 s at 46° C., 30 s at 72° C. followed by 10 extension minutes at 72° C. Subsequent amplifications were effected using the primer SL1 in a combination with each of the primers of Group 1 using the above conditions as amplification conditions.

A fragment of 650 by was obtained, cloned in the pCR2.1 and sequenced as described above.

In order to amplify the 5′ end, the protocol indicated by the supplier of the Kit was followed, using the primer 1 as for the synthesis of the cDNA and the primers 2as-3as and SL1 for the amplifications. A fragment of about 500 by was obtained, cloned and sequenced.

In order to obtain the cDNA encoding the whole gene, 1 μl of cDNA used for the reactions with the degenerated primers was amplified, with the following primers:

| | |
|---|---|
| 5′-ATGGCGTCGACTGAAGAC-3′ | (SEQ ID NO: 26) |
| 3′-TCAGACGATGTCATGGCA-5′. | (SEQ ID NO: 27) |

The amplification conditions used were the following: 40 cycles comprising 10 s at 98° C., 30 s at 60° C., and 1 minute at 72° C. followed by 10 extension minutes at 72° C. A fragment of about 1080 bases was obtained, cloned in the vector pCR2.1 and sequenced.

Cloning of the Receptor OR83b

In order to isolate the cDNA encoding the receptor OR83b of Spodoptera littoralis, a specific reverse primer and a degenerated forward primer were designed. The degeneration of the forward primer interested only one position since the alignment between the respective nucleotide sequences deriving from Spodoptera exigua, Heliothis virescens, Helicoverpa zea and Mamestra brassicae revealed a very high preservation degree among each other. The sequences of the primers are indicated hereunder:

| | | |
|---|---|---|
| Fw | ATGACCAA(AG)GTGAAGGCCCAG | (SEQ ID NO: 28) |
| Rv | GTGTTGGTACAACTCAAGTAA. | (SEQ ID NO: 29) |

The cDNA prepared as described in the previous paragraph was used, and of this 1 μl was amplified with 250 nM of each of the two primers in 30 cycles comprising 10 s at 98° C., 30 s at 50° C., and 1.5 minutes of extension. A fragment of about 1,500 by was obtained, which was cloned in the vector pCR2.1 and sequenced as described above.

Cloning of the Receptor DHR

In order to isolate the cDNA of the receptor DHR from Spodoptera littoralis, degenerated primers were designed on the basis of the alignment among the corresponding protein sequences of Manduca sexta, Drosophila melanogaster, Nilaparvata lugens e Acheta domesticus. The sequences of the primers used are the following:

(SEQ ID NO: 30)
1Fw:
TT(TC)(TC)T(ATCG)TA(TC)TT(TC)AA(AG)GA(ATCG)
(TC)T(ATG)(AC)G(ATCG)TG(TC)
(SEQ ID NO: 31)
2Rv:
A(AG)(TC)TT(ATCG)GT(ATCG)AT(ATCG)A(AG)(ATCG)
ACCCACAT(ATCG)AT.

RNA was extracted from V-phase larvae with the method already described. For the quantification of the RNA and synthesis of the cDNA, the protocols described for the cloning of AlstAR were used.

A fragment of 550 bp, was obtained through 45 amplification cycles, comprising 10 s at 98° C., 30 s at 48° C., and 1 minute at 72° C. followed by 10 extension minutes at 72° C., which was subsequently cloned in the vector pCR2.1 and sequenced.

On the basis of the sequence, specific primers were designed and used for the subsequent 3′ and 5′ RACE reactions following the method already described. The sequences of the primers are the following:

Group 1:

| | | |
|---|---|---|
| 1s | AACCTCATGTCGACGTATATTCTGTCT | (SEQ ID NO: 32) |
| 2s | ATGCTTGTAGAAGGTTTGTACCTGTAC | (SEQ ID NO: 33) |
| 3s | TGGGTTATATGCAGGTGCTTCGTCAAC | (SEQ ID NO: 34) |

Group 2:

| | | |
|---|---|---|
| 1as | GGATGGGGTGCGCCGGCGGTGTTCCTA | (SEQ ID NO: 35) |
| 2as | CATACATATGACCAGAATCGTACACGA | (SEQ ID NO: 36) |
| 3as | GGCGAGGTAGATGAGGCTGGTGACGTC. | (SEQ ID NO: 37) |

For 3′, a fragment of about 650 by was isolated through 40 amplification cycles, comprising 10 s at 98° C., 30 s at 46° C., 1 minute at 72° C. and followed by 10 extension minutes at 72° C. For 5′, a fragment of about 500 was obtained through 40 amplification cycles according to the procedure described above.

The following primers were used for the isolation of the entire cDNA:

| | |
|---|---|
| 5′-ATGGCGGAGAAGTGCCTGGCG-3′ | (SEQ ID NO: 38) |
| 3′-TCATACCGTGAGTCGTATGCT-5′. | (SEQ ID NO: 39) |

A fragment of 1190 by was amplified by 1 μl of cDNA, synthesized by RNA extracted from V-instar larvae, with the Fw and Rv primers through 45 amplification cycles, comprising 10 s at 98° C., 30 s at 55° C., 30 s at 72° C. and followed by 10 extension minutes.

Cloning of the Receptor LGR1

In order to isolate the cDNA of the receptor LGR1 from Spodoptera littoralis, degenerated primers were designed on the basis of the alignment among the corresponding protein sequences of Bombyx mori, Drosophila melanogaster e Aedes aegypti. The sequences of the primers used were the following:

(SEQ ID NO: 40)
1Fw:
GC(ATGC)TA(TC)(TC)T(AGCT)AC(AGCT)CA(TC)
(GC)(AGCT)(AGCT)TT(TC)CA(TC)TG(TC)TG(TC)
(SEQ ID NO: 41)
1Rv:
TC(AGCT)A(TC)(AGCT)GG(AGCT)A(AG)(AG)CA
(AGCT)AT(AGCT)(GC)(AT).

RNA was extracted from the eggs of Spodoptera littoralis, following the method already described. For the quantification of the RNA and synthesis of the cDNA, the protocols described for the cloning of AlstAR were used.

A fragment of about 1,250 bp, was obtained through 45 amplification cycles, comprising 10 s at 98° C., 30 s at 48° C., 1.5 minutes at 72° C. and followed by 10 extension minutes at 72° C., which was subsequently cloned in the vector pCR2.1 and sequenced.

From an analysis of the sequence, the following primers were designed, subsequently used for the 3′ and 5′ RACE reactions with the methods previously described:

Group 1:

| | | |
|---|---|---|
| 1s | TGATGAAAATGCCTTCGC | (SEQ ID NO: 42) |
| 2s | CACGAACCAGTCAACAACAC | (SEQ ID NO: 43) |
| 3s | GAAAGCGTACCTGACGCATCATTTCCA | (SEQ ID NO: 44) |
| 4s | GTTATCAGTAATTACTTTAACTATAGT | (SEQ ID NO: 45) |

Group 2:

| | | |
|---|---|---|
| 1as | ACTGCCGGAGTCTGAGAAGTA | (SEQ ID NO: 46) |
| 2as | GTACTCCTCGCTCGTAGTCG | (SEQ ID NO: 47) |
| 3as | CATTTCGACTGCATTATAGC | (SEQ ID NO: 48) |
| 4as | GCCTTCTAGGTCTATAGCTTG. | (SEQ ID NO: 49) |

A fragment of about 500 by and a fragment of 860 by were isolated, cloned in the vector pCR2.1 and sequenced at 3′ and 5′ respectively, through 35 amplification cycles on the cDNA synthesized starting from RNA extracted from the eggs of Spodoptera littoralis, according to the procedures described in the previous paragraphs.

In order to isolate the cDNA corresponding to the entire sequence encoding the receptor LGR1 from Spodoptera littoralis, the following primers were used, corresponding to the ends 5′ and 3′ respectively:

(SEQ ID NO: 50)
5′-ATGTATTGGAGATTATGTATTTGGGCT-3′
(SEQ ID NO: 51)
3′-TTAAAGCGGTACCTCACTACTGTCTTT-5′.

A fragment of 2,250 by was isolated, cloned and sequenced through standard amplification procedures.

Cloning of the Receptor Oct/TyrR

For the isolation of the cDNA encoding the receptor Oct/TyrR from Spodoptera littoralis, degenerated primers were designed on the basis of the alignment among the nucleotide sequences of the corresponding genes of Bombyx mori, Heliothis virescens and Mamestra brassicae.

The sequences of the primers used are the following:

| (SEQ ID NO: 52) | |
|---|---|
| 1Fw | CCAGAATGGGA(AG)GC(AT)AT(TC)TGCAC |
| (SEQ ID NO: 53) | |
| 2Rv | AC(AG)CCCATTAT(TG)AT(AG)CC(TC)AG(AG)G. |

The primers were used for amplifying a fragment of 1,090 by from cDNA synthesized on RNA extracted from heads of individual adults, prepared according to the methods described above. Once the fragment had been cloned in the vector pCR2.1 and sequenced, the following primers, used for the 3′ and 5′ RACE reactions, were designed:

Group 1:

| | | |
|---|---|---|
| 1s | CCAGAAAATTGACACCAA | (SEQ ID NO: 54) |
| 2s | GAGAGTAACTCGAAAGAAAC | (SEQ ID NO: 55) |
| 3s | TGCTGTTTATCAATTCATTGAAGA | (SEQ ID NO: 56) |

Group 2:

| | | |
|---|---|---|
| 1as | AGTTCTTTTTTTAGTGGCCAAA | (SEQ ID NO: 57) |
| 2as | GACAAGGCGTATCAGGTTC | (SEQ ID NO: 58) |
| 3as | ACCCTAGAAGTGGCGGAGAGCTAATA. | (SEQ ID NO: 59) |

A fragment of about 380 by and a fragment of 590 by were isolated, cloned in the vector pCR2.1 and sequenced at 3′ and 5′ respectively, through 40 amplification cycles on the cDNA synthesized starting from RNA extracted from heads of individual adults of Spodoptera littoralis, according to the procedures described in the previous paragraphs.

In order to isolate the cDNA corresponding to the entire sequence encoding the receptor Oct/TyrR, the following primers were used:

| | |
|---|---|
| 5′-ATGGGGCAAACAGCTACACAC-3′ | (SEQ ID NO: 60) |
| 3′ CTCTGTATGAAACCGTGA-5′. | (SEQ ID NO: 61) |

A fragment of 1,434 bp, was isolated, cloned and sequenced through 40 amplification cycles, comprising 10 s at 98° C., 30 s at 55° C. and 1 minute at 72° C.

Cloning of the Receptor PBANR

The sequence of the receptor PBANR was already available in data bank, consequently primers corresponding to 5′ and 3′ of the encoding region were designed, whose sequences are indicated hereunder:

| | |
|---|---|
| 5′-ATGACATTGTCAGCGCCCCCGATC-3′ | (SEQ ID NO: 62) |
| 3′-TCAATCATGAATGTAACA-5′. | (SEQ ID NO: 63) |

The primers were used for amplifying 1 μl of cDNA synthesized starting from RNA extracted from V-instar larvae, according to the procedures described. The amplification was effected for 40 cycles of 10 s at 98° C., 30 s at 55° C., 40 s at 72° C., followed by 10 extension minutes at 72° C. A fragment of 1,053 by was obtained, which was subsequently cloned in the vector pCR2.1 and sequenced according to the procedures described.

Cloning of Part of the Sequence Encoding the Receptor AlstCR

For the isolation of the receptor AlstCR of Spodoptera littoralis, the following degenerated primers were designed on the basis of the homology among the respective protein sequences of Apis mellifera, Drosophila melanogaster and Anopheles gambiae:

| | |
|---|---|
| | (SEQ ID NO: 64) |
| 1Fw: | GA(AG)TG(TC)TT(TC)(TC)T(AGCT)AT(ATC)GG(ATGC) |
| | (SEQ ID NO: 65) |
| 1Rv: | (ATGC)GC(AG)CA(ATGC)GT(AG)CA(ATGC)GC(TC)TT. |

The primers 1Fw and 1Rv were used for amplifying cDNA transcribed from RNA extracted from heads of individual adults, through 40 amplification cycles at 45° C. A fragment of 726 by was isolated, subsequently cloned in the vector pCR2.1 and sequenced.

Cloning of Part of the Sequence Encoding the Receptor AKHR

For the isolation of the receptor AKHR of Spodoptera littoralis, the following degenerated primers were designed on the basis of the homology between the nucleotide sequences of the corresponding receptors of Bombyx mori and Periplaneta Americana:

| (SEQ ID NO: 66) | |
|---|---|
| 1Fw: | GACCTGATGTGC(AC)G(AC)(AG)TCATG |
| (SEQ ID NO: 67) | |
| 1Rv: | GTC(AG)ATCCA(AG)TACCACAG(AG)CA. |

The primers 1Fw and 1Rv were used for amplifying cDNA transcribed from RNA extracted from heads of individual adults, through 40 amplification cycles at 45° C. A fragment of 534 by was isolated, subsequently cloned in the vector pCR2.1 and sequenced.

Cloning of the Receptors in the Plasmids pENTER and PH7GW1WG2

The genes encoding the receptors AlstAR, AlstCR, DHR, AKH, OR83b and Oct/TyrR were isolated from the plasmids pCR® 2.1-TOPO® by digestion or by PCR using a primer forward and a primer reverse homologous to the sequence of the gene to be isolated, carrying the recognition sequence for the restriction enzymes EcoRI (forward primer) and XhoI (reverse primer), compatible with the polylinker of the plasmid pENTR.

LGR1 receptor receptor was amplified by the plasmid pCR® 2.1-TOPO with a forward primer carrying the recognition sequence for BamHI enzyme and a reverse primer containing the XhoI site. For cloning PBAN receptor a forward primer containing the EcoRI site was used in combination with a reverse primer carrying the sequence cut by NotI enzyme.

The sequences of the primers are indicated hereunder:

(SEQ ID NO: 68)
AlstAR Fw
5′-GGAATTCCATGGCGTCGACTGAAGAC-3′
(SEQ ID NO: 69)
AlstAR Rev
5′-CCTCGAGGTCAGACGATGTCATGGCA-3′
(SEQ ID NO: 70)
AlstCR Fw
5′-GGAATTCCCTGCGTTCCATTCACTGCTA-3′
(SEQ ID NO: 71)
AlstCR Rev
5′-CCTCGAGGAAGAATTGGGTTCATGGCAG-3′
(SEQ ID NO: 72)
DHR Fw
5′-GGAATTCCGATGGCGGAGAAGTGCCTGGC-3′
(SEQ ID NO: 73)
DHR Rev
5′-CCTCGAGGGTCATACCGTGAGTCGTATGC-3′
(SEQ ID NO: 74)
AKH Fw
5′-GGAATTCCGGACCTGATGTGCCGAGTCATG-3′
(SEQ ID NO: 75)
AKH Rev
5′-CCTCGAGGGCTTGTCGATCCAATACCAC-3′
(SEQ ID NO: 76)
LGR1
5′-CGGGATCCCGGATGTATTGGAGATTATGTATTTGGGC-3′
(SEQ ID NO: 77)
LGR1 Rev
5′-CCTCGAGGGTTAAAGCGGTACCTCACTAC-3′
(SEQ ID NO: 78)
OR83b Fw
5′-GGAATTCCGATGACCAAAGTGAAGGCCCAGG-3′
(SEQ ID NO: 79)
OR83b Rev
5′-CCTCGAGGGTTACTTGAGTTGTACCAACACC-3′
(SEQ ID NO: 80)
Oct/TyrR Fw
5′-GGAATTCCGGGGCAAACAGCTACACACG-3′
(SEQ ID NO: 81)
Oct/TyrR Rev
5′-CCTCGAGGGTCACGGTTTCATACAGAGTAAC-3′
(SEQ ID NO: 82)
PBAN-R Fw
5′-GGAATTCCGATGACATTGTCAGCGCCCCCG-3′
(SEQ ID NO: 83)
PBAN-R Rev
5′- TAAAGCGGCCGCTCAATCATGAATGTAACAAAAA-3′.

The amplification reaction was performed in a final volume of 50 μl using 50 ng of plasmid DNA, 200 μM of dNTPs, 0.25 μM primer forward, 0.25 μM primer reverse, 5× Phusion HF buffer, 1 U of Phusion High-Fidelity DNA Polymerase (Finnzymes). The amplification program consists in a denaturing cycle at 98° C. for 30 s, 35 cycles at 90° C. for 10 s, 52° C. for 30 s, 72° C. for 1 min and one final extension cycle at 72° C. for 10 min. About 5 μl of amplified product were separated on agarose gel at 1% to verify its quality and molecular weight. After purification from the reagents used for the amplification (GenElute™ PCR Clean-Up Kit, SIGMA) the remaining PCR reactions were subjected to digestion with 20U of appropriate restriction enzymes to produce sticky ends for the subsequent cloning in the vector pENTR, also digested with the same restriction endonucleases. The ligation reaction between the genes of the receptors (200 ng) and the vector pENTR (300 ng) were performed in a volume of 20 μl at 16° C., using 20u of T4 DNA ligase (Biolabs). 10 μl of ligase reaction were used for transforming competent cells of E. coli DH5a plated on LB with the antibiotic kanamycin (50 mg/l to select the cell clones carrying the pENTR plasmids. The colonies obtained were inoculated with liquid LB and kanamycin at 37° C. for the whole night.

The plasmid DNA was extracted (Wizard® Plus SV Minipreps DNA Purification System, Promega) and digested with 10u of suitable restriction enzymes.

The pENTR plasmids with the cloned receptors were used for the recombination reaction with the binary vector pH7GW1WG2(I). 150 ng of pENTR plasmid were incubated with 300 ng of pH7/GW1WG2(I) at 25° C. in the presence of the enzyme Gateway® LR clonase II Enzyme Mix (Invitrogen) for two hours. The reaction was then blocked with Proteinase K at 37° C. for 10 min. and used for transforming the DH5α cells. The plasmids were analyzed by digestion with restriction enzymes and further verified by means of sequence analysis. Competent cells of Agrobacterium tumefaciens (C58) were transformed with the recombinant plasmids with the freezing/defrosting technique. About 10 μg of plasmid were added to 100 μl of competent cells and incubated in ice for 5 minutes. The cells were put in dry ice/ethanol for 5 minutes and then transferred at 37° C. for 10 minutes. 1 ml of LB was added to the cells and they were incubated at 28° C. for 3 hours before being plated on LB containing Rifampicin 100 mg/l and Spectinomycin 100 mg/l.

The resistant colonies were analyzed by means of PCR with primers specific for each cloned receptor.

Transformation of Tobacco Plants with dsRNA

The Gateway technique was used for the expression of the dsRNA in tobacco plants: the sequences encoding the receptors were isolated from the pCR® 2.1-TOPO® plasmids in which they were cloned and sequenced. The PCR reactions were effected using oligonucleotides complementary to the gene sequence and carrying the sites recognized by the restriction enzymes for the cloning of the plasmid pENTR (Invitrogen). This plasmid is an Entry Vector which, in addition to the cloning of the DNA sequences, allows them to be transferred into the expression vector (Destination Vector) thanks to the presence of two specific recombination sites (attL1 e attL2) which flank the cloned PCR product. The recombinant clones were used for the recombination reaction with the binary vector pH7GWIWG(I) (Plant System Biology) for the plant expression. This plasmid is characterized by the presence of the promoter 35S for the constitutive expression of the gene cloned downstream of the sites attR1 and attR2, which serve for recombination with the Entry Vector and transfer of the 5′-3′ strand of the cloned gene upstream of an intronic sequence and downstream of another identical intronic sequence. The presence of the constitutive promoter ensures the expression of the gene whereas the intronic sequence allows the formation of the dsRNA. The recombination reaction between the sites attL1 and attL2 of pENTR and the sites attR1 and attR2 of pH7GWIWG(I) is mediated by the enzyme Gateway® LR Clonase™ (Invitrogen). The recombinant plasmids were characterized by means of digestion with restriction endonucleases and further verified by sequencing. These plasmids were transferred into competent cells of Agrobacterium tumefaciens (strain C58) for the transformation of tobacco plants. For each construct, about 50 leaf disks were co-cultivated and the same quantity co-cultivated with agrobacterium transformed with the empty plasmid pH7GWIWG(I) as control for the subsequent biological tests.

After transformation of Nicotiana tabacum plants with Agrobacterium tumefaciens carrying the plasmid pH7GW1WG2 expressing the single cloned receptors, the leaf explants were co-cultivated for 3 days with 100 μl of bacterial culture grown at 28° C. until reaching a DO₆₀₀=0.8 in the presence of 10 ml of liquid medium A10 (3.6 g/l B5, 250 mg/l NH₄NO₃, 500 mg/l MES, 2% Glucose, 1 μg/ml benzylaminopurine, 0.1 μg/ml naphthalene-acetic acid, pH 5.7). The Agrobacterium was removed by rinsing the pieces of leaf with fresh A10 medium containing cefotaxime 500 μg/ml. The leaf disks were then transferred onto solid A11 medium in the presence of the antibiotic hygromycin (30 mg/l). The explants were transferred every week onto fresh medium until the formation of calluses which were put on A12 (A11 with a concentration of cefotaxime equal to 200 μg/ml). The shoots were transferred onto solid MS30 (Murashige and Skoog 4.4 g/l, Saccharose 30 g/l pH 5.7) and hygromycin.

Characterization of the Transgenic Plants

The genomic DNA was extracted from 0.5 g of leaves of tobacco plants grown on selective medium (GenElute™ Plant Genomic DNA miniprep Kit, Sigma) and used as a mould for verifying the presence of exogenous DNA by means of PCR. The plants positive to PCR were further characterized for the presence of dsRNA by extracting the RNA from the leaves of the transgenic plants (GenElute™ Mammalian Total RNA Miniprep Kit, Sigma) for the reverse-transcription of the cDNA. For this purpose, 2 μg of total RNAs were incubated with 250 ng of Random hexamers (Invitrogen) at 70° C. for 5 minutes and then in ice for 5 minutes. A solution containing 1 mM of dNTP, 1× Reaction buffer, 20 u of Ribonuclease inhibitor was added to the reaction and the mixture was incubated at 25° C. for 5 minutes. 200 u of RevertAid™ M-MuLV Reverse were added to the samples and the reaction incubated at 25° C. for 10 minutes, then at 42° C. for 60 minutes and then for 10 minutes at 70° C.

1 μl of cDNA is used for the PCR reactions and for each receptor the primers used for cloning in pENTR.

Tests with larvae of S. littoralis

For the biological tests on with dsRNA/AlstC-R and dsRNA/DHR transgenic plants, instar-III larvae of Spodoptera littoralis were used. Each larva was placed in a container having a diameter of 6 cm in which pieces of leaf of each transgenic line were introduced daily. As control, larvae of the same age were fed with leaves of plants transformed with the empty plasmid, and thus not expressing any dsRNA.

RESULTS

The plants regenerated on a selective medium were characterized for the presence of the heterologous gene by means of PCR on genomic DNA extracted from the leaves and the production of the dsRNA was verified by means of RT-PCR on cDNA reverse-transcribed from total RNA. FIG. 3 shows the RT-PCR analysis of the dsRNA on AlstC-R transgenic plants. In all the 7 lines analyzed, the expression level of dsRNA was quite high, while in the control plants (WT1 and WT2) no expression was detected. To make sure the amplification reaction was working correctly, a positive control (C+) of a plasmid containing the AlstCR gene was used as template. Analogous results were obtained for the plants expressing the dsRNA of other receptors (AlstA-R, DHR, PBAN-R, OctR). All the positive transgenic plants were micropropagated by nodal cuttings in order to obtain homogeneous populations of single transformants to be tested with the larvae.

The tests with larvae were carried out using instar-I larvae of S. littoralis fed on pieces of leaf of transgenic plants grown in sterile conditions. The larvae were raised together until instar III and then each one was isolated in 10 cm-petri dish where pieces of leaves were added daily for the whole life cycle of the insect until reaching the pupa phase.

In a first preliminary experiment, 20 larvae for each transgenic line, were used in the test. The graph in Figure shows the result obtained from the biotest on plants expressing dsRNA for AlstC-R. The transgenic lines analyzed (C2, C3, C5a and C7) are indicated on the axis of the abscissa whereas the mortality percentage on the ordinates. It is evident that the larvae fed on transgenic plants show a much higher mortality percentage with respect to the larvae fed on the control plants (WT). While the larvae fed on the control showed a mortality rate of around 3%, those fed on the transgenic lines had around 80% mortality (lines AlstC-R 5a and 7) and 100% (lines AlstC-R 2 and 3), after 4 weeks of feeding.

In a second experiment, the mortality rate of larvae fed on two transgenic lines (expressing dsRNA of AlstCR and DHR, respectively) was tested and compared to control larvae fed on wt plants. The mortality rate of the larval population was measure over 10 days, starting from instar I larvae. In the graph showed in FIG. 5, the mortality rate of the larvae fed on both the transgenic lines reach 100% after 8 days, while in the control is still 60%. This experiment clearly indicated that the presence of dsRNA, both AlstCR and DHR, in the plants produced a significant reduction of the vitality of the larvae, confirming the validity of the technology proposed.

### EXAMPLE 2

### Transformation of Tobacco Plants, in Order to Protect them from the Nematode Meloidogyne incognita

Materials and Methods

Selection of the GPCR Targets

The GPCR receptors of M. incognita which were cloned from this organism and selected as targets of the present embodiment of the invention are the following:

1. a homologue of the serotonin receptors of C. elegans, called MiSerR1-like (FIG. 2; SEQ ID NO:9);

2. a homologue of the CeDop-1 dopamine receptor of C. elegans, called MiDopR1-like (FIG. 2; SEQ ID NO:10);

3. a homologue of the CeNPR1-like neuropeptide receptor of C. elegans, called MiNPR1-like (FIG. 2; SEQ ID NO:11);

4. a homologue of a putative GPCR of C. elegans (T27D1.3) (belonging to the family of Rhodopsin-like GPCRs), called MiRho1-like;

5. a homologue of a putative GPCR of C. elegans (T07D4.1) (belonging to the family of Rhodopsin-like GPCRs), called MiRho2-like;

6. a homologue of a putative GPCR of C. elegans (B0563.6) (belonging to the family of Rhodopsin-like GPCRs), called MiRho3-like;

7. a homologue of a putative GPCR of C. elegans (F02E8.2) (belonging to the family of Rhodopsin-like GPCRs), called MiRho4-like.

In addition to these 6 receptors, another sequence corresponding to a GPCR of interest was identified in a data bank: MiNpR1-like (Q2TGX5_MELIC) which is a homologue of a neuropeptide receptor of the model nematode C. elegans (FIG. 2, SEQ ID NO:11).

Tobacco plants were transformed with plasmids containing gene sequences (which produce dsRNA in plants) of the receptors MiSerR1-like (SEQ ID NO:9), MiDopR1-like (SEQ ID NO:10) and MiNpR1-like (SEQ ID NO:11). The nucleotide sequences of the receptors which were analyzed are indicated in FIG. 2.

Cloning of the Receptors of M. incognita

In order to clone the receptors MiSerR1-like and MiDopR1-like from M. incognita, the data bank of the expressed sequences of the nematode M. incognita (available at http://www.nematode.net/BLAST/Cluster.BLAST /index.php) was interrogated with various peptide sequences of the homologous receptors of C. elegans.

The clones identified were:
AW 735607 for MiSerR1-like
AW 571066 for MiDopR1-like.

Starting from the sequences of the identified clones, specific oligonucleotides were designed, and used for amplifying portions of the receptor sequences. The sequences of the whole receptors were then obtained using the 5′ and 3′ Race method, previously described. With respect to the MiNpr1-like receptor, the cloning was performed using specific primers at 5′ and 3′ of the gene whose sequence was already present in data banks Q2TGX5_MELIC). The oligonucleotides used for the amplification are the following:

| For MiSerR1-like (SEQ ID NO: 9): | |
|---|---|
| (SEQ ID NO: 84) | |
| MiserR F1: | GATTTGGAAAATTTGGACGAT |
| (SEQ ID NO: 85) | |
| MiserR F2: | GGAGGGTCTTTTGTCCATGCA |
| (SEQ ID NO: 86) | |
| MiserR F3: | TCTCATCCAATAATTGCAATT |
| (SEQ ID NO: 87) | |
| MiserR R1: | ACGTAATGCTGAATATCGAAG |
| (SEQ ID NO: 88) | |
| MiserR R2: | CAAATTTAAAATTGAAGCAGT |
| (SEQ ID NO: 89) | |
| MiserR R3: | AGCCAAAGCAAGTGATATAAG |
| For MiDopR1-like (SEQ D NO: 10): | |
| (SEQ ID NO: 90) | |
| MidopR F1: | ACTCTTGGTGTTATTATGGGC |
| (SEQ ID NO: 91) | |
| MidopR F2: | TGGCTAGGTTATGCCAATTCT |
| (SEQ ID NO: 92) | |
| MidopR F3: | CGAGACTTTCGACGTGCCTTT |
| (SEQ ID NO: 93) | |
| MidopR R1: | AAAGGCACGTCGAAAGTCTCG |
| (SEQ ID NO: 94) | |
| MidopR R2: | AGAATTGGCATAACCTAGCCA |
| (SEQ ID NO: 95) | |
| MidopR R3: | GCCCATAATAACACCAAGAGT. |

The following oligonucleotides were used for the amplification of the whole encoding region:

| (SEQ ID NO: 96) | |
|---|---|
| MiserR Forward: | ATGTTAGAAAATGATTTGGAAAA |
| (SEQ ID NO: 97) | |
| MiserR Reverse: | TTATTTTGATGATTCCATCA |
| (SEQ ID NO: 98) | |
| MiDopR Forward: | ATGTTGCCCTGGTGGCTACCTCT |
| (SEQ ID NO: 99) | |
| MiDopR Reverse: | TTAAAAACATAAAAATCTCA |
| (SEQ ID NO: 100) | |
| MiNprR Forward: | ATGGAAGCATCTACAATGGAATT |
| (SEQ ID NO: 101) | |
| MiNprR Reverse: | TCATATCCTCTCATCTGTAT. |

The receptors thus amplified were cloned in the vector PCR2.1 (Invitrogen) and sequenced.

Analogously to what is described in Example 1 for the genes encoding the GPCR receptors of Spodoptera littoralis, the cDNAs of M. incognita were transferred into the plasmid pENTER and subsequently into the binary vector for plants PH7GW1WG2.

The trangenic lines expressing dsRNA corresponding to MiNPR1-like of M. incognita were used for root infection tests.

Root Infection Experiments

Two weeks old tobacco plants were transferred into 16 cm-pots containing sterile soil mixed with around 5-6 M. incognita galls, previously excised from the roots of other infected plants. After 6 weeks, the soil was removed and the plant roots analyzed for the presence of galls. It was measured the amount of total biomass infected of the transgenic plants and the values compared to those of WT control uninfected plants.

Results

As described above, tobacco plants belonging to 3 different transgenic lines expressing dsRNA of NPR1-like were infected with M. incognita worms. After 3 weeks, it was estimated the percentage of infected roots of the transgenic plants and compared to that of untransformed control plants (WT). As shown in FIG. 6, the roots of the transgenic plants showed a reduction in the infection level compared to WT plants. This result indicates that the transgenic plants, expressing the dsRNA of M. incognita NPR1-like gene, are more resistant to the nematode infection, confirming that the technology here proposed find applications also in the protection of plants against nematode attacks.

### BIBLIOGRAPHY

Aguilar R, Maestro J L, Vilaplana L, Pascual N, Piulachs M D, Bellés X (2003). Regul Pept. October 15; 115(3): 171-7.

Baum J A, Bogaert T, Clinton W, Heck G H, Feldman P, Ilagan O, Johnson S, Plaetinck G, Munyikwa T, Pleau M, vaughn T Y and Roberts J (2007). Nature Biotech, 25 (11): 1322-1326.

Brantl, S. (2002) Biochimica et Biophysica Acta. 1575: 15-25.

Farooqui T (2007). Neurochem Res. 32: 1511-1529.

Hamilton, A. J. and Baulcombe, D. C. (1999). Science 286: 950-952.

Hammond, S. M. Boettcher, S., Caudy, A., Kobayashi, R. and Hannon, G. (2001). Science 293: 1146-1150.

Johnson E C, Bohn L M and Taghert P H (2004). Journal of Exper Biol, 207: 743-748.

Kusaba, M., Miyahara, K., Lida, S., Fukuoka, H., Takario, T., Sassa, H., Nishimura, M., Nishio, T. (2003) Plant Cell 15: 1455-1467.

Li L, Wright S J, Krystofova S, Park G, Borkovich K A (2007). Annu Rev Microbiol., 61: 423-452.

Meyering-Vos M, Merz S, Sertkol M, Hoffmann K H (2006). Insect Biochem Mol Biol. June; 36(6): 492-504.

Neuhaus E M, Gisselmann G, Zhang W, Dooley R, Stortkuhl K Hatt H (2005). Nature Neurosc, 8 (1): 15-17.

Nishi S, Hsu S Y, Zell K and Hsueh J W (2000). Endocrinology, 141 (11): 4081-4090.

Ogita, S., H., Uefuji, Y., Yamaguchi, N. Koizumi and Sano, H. (2003). Nature 423: 823.

Rafaeli A, Bober R, Becker L, Choi M Y, Fuerst E J and Jurenka R (2007). Insect Molec Biol, 16 (3): 287-293.

Staubli F, Jorgensen T J D, Cazzamali G, Williamson M, Lenz C, Sondergaard L, Roepstorff P and Grimmelikhuijzen J P (2002). PNAS, 99 (6): 3446-3451.

Tomoyasu, Y. and Denell, R. E. (2004). Dev. Genes Evol. 214: 575-578.

Weaver R J, Freeman Z A, Pickering M G, Edwards J P. (1994). Comp. Biochem. Physiol. C 107: 119-27.

1. Method for the preparation of transgenic plants resistant to the attack of one or more phytopathogens by RNA interference comprising the following steps : a) isolation of the cDNA nucleotide sequence encoding a G protein coupled receptor (GPCR), or a portion thereof, whose function is vital for the phytopathogen; b) construction of an expression vector comprising the cDNA nucleotide sequence encoding a GPCR receptor or a portion thereof as determined by step a), flanked by two specific recombination sites,-c) recombination reaction of the expression vector of step b) with a binary vector comprising a constitutive or tissue-specific promoter, the same specific recombination sites and an intronic sequence to obtain a recombinant plasmid; d) transfer of said recombinant plasmid into competent cells of Agrobacterium tumefaciens and transformation of the plant; e) growing the plant and obtaining the constitutive or tissue-specific expression of dsRNA in said plant.

2. Method according to claim 1, wherein said phytopathogens are selected from herbivorous insects, nematodes and fungi.

3. Method according to claim 2, wherein said herbivorous insect is selected from Spodoptera littoralis, Heliotis virescens

4. Method according to claim 2, wherein said nematode is selected from Meloidogyne incognita, Meloidogyne hapla, Globodera rostochiensis.

5. Method according to claim 1, wherein when the dsRNA expression is tissue-specific, said tissue is selected from the group consisting in phylloplane, root system, trunk, buds, flowers, fruits.

6. Method according to claim 1, wherein said GPCR receptor is selected from the group consisting in allatostatin A or C, octopamin/tyramine, PBAN peptide, diuretic hormone, adipokinetic hormone, neuropeptides, dopamine, serotonin or rhodopsin-like receptors.

7. Method according to claim 6, wherein when said insect is Spodoptera littoralis the cDNA nucleotide sequence encoding a GPCR receptor or a portion thereof is selected from the group consisting in AlstAR (SEQ ID NO:1), AlstCR (SEQ ID NO:2), DHR (SEQ ID NO: 3) , AKHR (SEQ ID NO:4), LGR1 (SEQ ID NO:5), Oct/TyrR (SEQ ID NO:6), OR83b (SEQ ID NO:7) and PBAN-R (SEQ ID NO: 8) .

8. Method according to claim 6, wherein when said nematode is Meloidogyne incognita the cDNA nucleotide sequence encoding a GPCR receptor is selected from the group consisting in serotonin-like receptor (SEQ ID NO: 9), dopamine-like receptor (SEQ ID NO.-10), neuropeptide receptor (SEQ ID NO: 11), rhodopsin-like receptors.

9. Method according to claim 1, wherein steps a)-c) may be repeated in parallel using in each step a cDNA nucleotide sequence encoding a GPCR receptor which is vital for different phytopathogens for obtaining one or more recombinant plasmids with which d) co-transforming the same plant or d1) transforming different plant and cross them.

10. Method according to claim 1, wherein said plant belongs to the family of Solanaceae, preferably tobacco.

11. Transgenic plant resistant to one or more phytopathogens obtainable according to the method as defined in claim 1.

12. Transgenic plant according to claim 7, that is a tobacco plant resistant to Spodoptera littoralis.

13. Transgenic plant resistant to Spodoptera littoralis characterized in that it is a tobacco plant and expresses a dsRNA wherein the antisense strand is complementary to the mRNA encoding for SEQ ID NO: 2 and/or SEQ ID NO: 3.

14. Transgenic plant resistant to Meloidogyne incognita characterized in that it is a tobacco plant and expresses a dsRNA wherein the antisense strand is complementary to the mRNA encoding for SEQ ID NO: 11.

15. Method for the preventive treatment of plants to make them resistant to the attack of one or more phytopathogen insects, nematodes, fungi comprising the steps a) -e) as defined according to claim 1.

16. Method according to claim 15, further comprising a step for treatment of the plants thus obtained with fertilizers, biostimulating agents, fungicides, synthesis or natural nematocides, herbicides or insecticides.


SEQUENCE LISTING (101 sequences)

SEQ ID NO: 1 - DNA, 1086 bp, Spodoptera littoralis
  misc_feature (1)..(1086): Allatostatin A receptor encoding cDNA
atggcgtcgactgaagacgaatttaggagcatatgccttaacttaaccagtgaggaggctatgcttggaggatgtaactacagcagcgaatatgaaaatggagaactgttggagaaggtcgtctcgagggttgtgcccatcttctttggcttcataggcatcgttggattagttggcaatgctttggtcgtcttagtggtcgctgccaaccctggcatgcggtccactaccaacctgctcatcataaacttggctgtcgctgacttgctctttgtgatcttctgcgttccattcactgctaccgactatgtcatgccacgatggcccttcggagactggtggtgcaaggttgtccagtacttcattgtagtcactgctcatgcgtccgtctacactctagtactcatgtctctagacaggttcatggctgtagtgcatcccatagcttccatgtctatcagaacagagaagaacgcattacttgctatagcttgcatatgggtggtgatattaaccacagctattcccgtgggcatctgccacggggagcgggaatactcctacttccatagaaaccattcctcctgcgtgttccttgaggagcaaggatatagcaagctagggttccagatgtctttctttttgtcttcctatgtgattcccttagctttgattagtgtattgtatatgtgtatgctgacgaggttgtggaagagtgctccaggagggagggtgtctgcggagagtcggagggggaggaagaaggtgacgagaatggttgtggtcgtagtggtggtgttcgcagtgtgctggtgcccaatacagataatcctgttggtaaaggcgctggacaagtaccacatcacatacttcacagtcactgcgcagatagtgtcgcatgtactagcctacatgaacagctgcgtcaacccagtgctgtacgccttcctctctgagaacttcagggtcgccttcaggaaggtaatgtactgcccgcccccctacaatgacggcatgtcaggccggccccagcccaccaagacgactcgtacaggcaatgggaactcctgccatgacatcgtctga

SEQ ID NO: 2 - DNA, 726 bp, Spodoptera littoralis
  misc_feature (1)..(726): Allatostatin C receptor encoding cDNA
tgctttcttatcggcatcccattcctgatcacaacgatgtctcttcgtgcgtggccgtttggacgcttcatgtgtaaggcctacatgatatctaccggaatcaaccagttcaccagctctatattcttgtgtataatgagtgcggatcgatacatagccgtgtgtcatccgatagccgcgccgcgactgcggacgccattcgtctctagagtggtgtcagctgcagcatggacagcgtcagctttggtgatgacgcctatatttatgtacacgacactcataccaacaccccatggtctgtcgtgcaatatagtgtggcccgagcaagagttcaataaaggccagacgtccttcactctgtactcgttcgctctcggtttcgccgcaccactcacccttatcttcgtgttttactgtctcgtgattcgcaaactgaagactgtcggccctaagaacaaatctaaagagaaaaagcgatcacacagaaaagtaaccaaattagtgctgactgtcattgctgtgtacgtgctgtgctggctgccgtactgggcgttccaggtagctctcatttactctccaccgaacgagtgcgccagccagatcactatcaccgtttttctggtagccgcgtgcttcagttacagtaactctgccatgaacccaattctttacgcctttctttctgacaacttcaagaaaagtttcctaaaggcctgcacctgtgcc

SEQ ID NO: 3 - DNA, 1191 bp, Spodoptera littoralis
  misc_feature (1)..(1191): DHR receptor encoding cDNA
atggcggagaagtgcctggcgcagaacgaagggctggcggaggaggagttctgccccgcgtactacgacgggctgctgtgctgggacgcgacgccctggaacacgctggcggtgcagcgctgcttcagcgagttccacggcgtccagtacgatgacacacagaacgcatcgcggatgtgcttggacggcgtgtggcacaactactcaaactacgcgaactgcacagaacgcatgaccaacgtgtcgccgactgacgtcaccagcctcatctacctcgccggctactcgctcagcctcgccgtgctttcactcgccgtcttcgtcttcctttatttcaaggacctgcgatgtctaaggaacacgatacataccaacctcatgtcgacgtatattctgtctgcctgcagctggatcttaaacttagctttacaaaattggtccgatgaggcccaacaagatcaaacctcgtgtacgattctggtcatatgtatgcactacttttacctcactaatttcttcgggatgcttgtagaaggtttgtacctgtacatgctcgtggtggagacgttcaccgctgaaaatattaaactgaaggtgtacactaccataggatggggtgcgccggcggtgttcctaacaatatgggttatatgcaggtgcttcgtcaacatcgtgccttccaaccctgctgatgggcttgccctggcaggtgaggctaagatgtgtatttggatgtcggagcaccaggtggattggatccataaagctccagcactcgctgggctcgccctcaacctcttcttcctcattaggattatgtgggtattaatcacaaaactgcggtctgcaaacacgttggagacggagcagtatcggaaagcaacgaaggcacttctagtcctgatcccactgctgggcatcaccaacctgctggtgctgtgtgggcccaatgacgactcctggttcgcacacgccttcgactacacgcgagcactcatgctttcaacacagggtttcttggtggctttgttctactgcttcatgaatacagaagtacggcacgcaatcagataccacgtcgagaggtggaagactgggaggaccatcggtggaggaaggaggagaggagcttcctactccaaggactggtctcctcggtcacgaacagaaagcatacgactcacggtatga

SEQ ID NO: 4 - DNA, 534 bp, Spodoptera littoralis
  misc_feature (1)..(534): AKHR receptor encoding cDNA
gacctgatgtgccgagtcatgatgttcaccaggaccttcggactgtacctctccagcttcgtgcttatctgtatagctgttgatagatactacgccattttaaagcctttgaacgtgacatgggaggcgcgcgtgcgcagagcgctgattatcgcgtgggtgtgcgccggcctcgccagcctgccgcagagcttcatattccacgtagaggagcatcctgatgttaaggggtactaccagtgcgtgtcgtacgggtcgttgccgacggagtgccacgagtttgcgtacttcttggtaaacatgatcctcatgtacgtcatccctctgctgtccacgctgtactgctcctcggcagctttgttggaaatcattcggagatctaacacttctaatgataagatgcgacggagcggcgtgggtatactcgggcgggcgcgggctcggacactcaagatgacagtcactatagttctagtcttttttacatgttggtcaccctactactgttattgtctgtggtattggatcgacaag

SEQ ID NO: 5 - DNA, 2250 bp, Spodoptera littoralis
  misc_feature (1)..(2250): LGR1 receptor encoding cDNA
atgtattggagattatgtatttgggctgttgcctttattttactggggagcagagaagcagcatgccactcgatgagtgagagtgtgtctgtggtgctgcaccagaccaacatcacacggctcacggcaaaggtcatccatgctgcgggctaccatttgcatcgagttgagcatttatcaataatggatgcaccaaaattggatcacatagtagtggacgatctgactaagatgcctaatctcaaatctctgtttataacgcaagctccgcgactacacagggtgtcgcccctccctccgctgcccgagctacgcacattcatcatcacaacctccggcttggtggaagtccccaacctgagccacgtgcacgacaagaagaataacaactcactcgcctatctgcaagctatagacctagaaggcaaccacataaagcgcctatcgccgcacgcattgcgcgtgcgcgctgatcaagtgtccttgaactacaacttgctagaagaagtgcctgcttttgcgttcaagaatgcgcagatttcaaaattgagtttcagaggaaacacgaaactaaaacatattgatgaaaatgccttcgcatcgaatttgctcctccgacaattagatttaagtaacaccgctattacctccctgcctacagcaggactacagaacttggaaacccttagaatagaaaagacaccttcattgaaatacatcccatcgatttacgaatttcagagtctccagaaagcgtacctgacgcatcatttccactgctgcgcgttccacttccccgagcgacacaacccaacgagacacaagctctatgaaacgcaaattgctataatgcagtcgaaatgtgcgagcgctagtcagcagaagaaaccagtagccagaagaaagaagaggtcactggcaccactcaagatcgttatgcctcagcgtaaaaacgatactacagactcattagaggaagacctgtcaacagcgactacgagcgaggagtacgacgctaacttgatggacgagtacttctcagactccggcagttgggaggacgaggacgaccagggagagttccacgaaccagtcaacaacacgctcggggccggcttcgcgatcatatccgctgagtgcgggaacttcagtattagcccacgaaaagtggaatgctttcccgcacctgatgcactgaatccgtgcgaagatgtgatgggctggtcgtggctccgagcgagtgtgtggttcgtcatcacggcggcggtggtgggcaacgttgccgtcctgctcgtcttacttacaaaccacacggagattacagtcccgcggttccttatgtgccacctcgccttctctgacctctgcaccggcctctacctgtttatgttggccgtcgtagacctcagatcttatggagaattcttcaactacgcctacgattggcagtacggggtcggctgcaagatcgctggctttctatcagtattttctggacagttatcagtaattactttaactatagtcacattggagcgctggttcgccataacttatgcaatatacttagaaaggaggatctctctatcagccgcagctaagataatgatgggcggttggttgtactccttcctgatggctggcttgccgctggtcggcgtgtcagactattcgtctacgtcaatatgtttaccggttgagagcaaggatgtgggagatgctgtgtaccagggatcaatatttatagtgagtgcgttggcgtgggtcacgattgtggtgtgctacgtgcagatatacaggtcgctagggggtggagagagttacggagggaggggggcggccgcagccgctgagagacgaattgcgaataaaatggcactcctcatcggaaccgacctgttgtgctgggcgcctgtggcgttcttcggcatcacagctctagcaggagtgccgctagtcgatgtcagtcacgggaaagtgttgctagtattcttttaccctctgaacgcttgtgcgaatccattcttatacgccatattaacgaaacaataccgaagagatttgataacgttgatagcacggacgggtcactgcacctggttagtggagaagtacaagttggcggcgaccccgccccccaccgcgcacactaacccttctgcgccggcgcagttgatgccgctagtagactccgtcacgtatcaaaggagccagtcacagtgtaaagacagtagtgaggtaccgctttaa

SEQ ID NO: 6 - DNA, 1437 bp, Spodoptera littoralis
  misc_feature (1)..(1437): OctR receptor encoding cDNA
atggggcaaacagctacacacgtaaacgcaaattacacattaattaattatactgatgaaataattgaggatgacagagacgcttgtgccgtcgcagatgaccctaaatatccgagtagtttcggtataacacttgcggtaccagaatgggaagcaatttgtacagccatagtcctaacgcttataataatctcaacgatagttggaaacattttggtgatcttgagtgtgtttacatacaaaccccttaggattgtccagaacttcttcatagtatcacttgctgtggcggatttgacagtagcaatattagtattgccactaaatgtggcatactccattctaggacaatgggtatttgggatttacgtatgcaaaatgtggttaacatgcgacataatgtgttgcacttcgtccattttaaatttatgtgcaatcgcattagacaggtattgggcgataaccgatcctataaattatgcacaaaagagaactttagaaagagtgatgttaatgattggagtagtttgggtactttcattagttattagctctccgccacttctagggtggaatgactggcccgacgtctttgaacctgatacgccttgtcgtttaacttcccaacctggcttcgttatcttctcttcatctgggtccttctacataccgctagttataatgactgtagtttattttgaaatatatttggccactaaaaaaagacttagagatcgcgctaaagccacaaagatcagcactatttctagcggtcagaatagatacaataataaagacgataacaatgatcatgatcaagattctgttagctcagaagccaatcacaatgagcaccaaggcgtgacacggttggcatcagacaatgagaaaaagaaaagaaccagaaaattgacaccaaagaaaaaaccaaagcgacgatattggagtaaggacgataaatcgcaaaacaaactgataataccgattctctctaacgataattcggtaactgatatgggtgataatttagaaaatagaaacacatcatcagagagtaactcgaaagaaactcacgaggatgatttgattgaagttactgagcctgcgccagtgaagagtcacaagcggccgaagcctaaccagcaaagtgctgtttatcaattcattgaagaaaagcaaaggatttccttgacacgtgagcgtcgagccgccagaacccttgggattataatgggcgtatttgtcgtgtgttggttgcccttctttgtcatataccttgttattcccttctgtgcgagttgctgtttgtccaacaagtttatcaatttcataacttggcttggctactgcaattctgcactcaaccctctaatatacaccatctttaacatggactttaggagagcattcaaaaagttactctgtatgaaaccgtga

SEQ ID NO: 7 - DNA, 1419 bp, Spodoptera littoralis
  misc_feature (1)..(1419): Or83b receptor encoding cDNA
atgaccaaagtgaaggcccagggcctcgtctccgacctgatgcccaacatcaagttgatgcaggcagctggacacttcctcttcaattaccatgcagaaaatggtggcatgagcggcctccttcggaagatctacgccagtacccacgcgattctcatcaccatacattttgcctgtatgggtatcaacatggcccagtactccgatgaggtcaacgagctgactgctaacactatcaccgtcctcttcttcactcacaccatcatcaagttggggttcttcgctttgaactccaagagtttttacaggactttagcagtatggaaccagtcgaacagccaccctctgttcacggagtcagatgcccgctaccatcaaattgcgctcaccaagatgaggagactcctgtacttcatctgcggcatgactgtcctgtcagttgttagctgggtcacactcacattcttcggtgaatcagtccgcctgattactagtaaggaaacgaatgagacgttgacagaagtagcccctcgattgcctctgaaggcctggtaccctttcaatgctatgagcggcaccacgtatattatcgctttcgctttccaggtctactggctcctcttctcaatggctatagcaaatctgatggatgttatgttctgctcctggctgatcttcgcttgtgaacagctgcagcacctgaaggccatcatgaaacctctcatggaactcagtgcttcactggacacgtatcggcctaatactgctgagctgttccgtgcttcttccactgagaaatcagaaaagattcctgatacagtcgacatggacatccgcggcatatattccacacagcaagatttcgggatgacgttgcgaggagctggaggtagactgcaggcctttggacagcagaacaacaaccctaatggcttgactccgaagcaggagatgctggcgaggtctgctattaagtattgggtggagaggcacaaacacgtcgtcagattggtagcatcgataggagacacgtatggtactgccctgctgttccacatgttggtgtcaactataacactcactctattggcttaccaggctactaagatcaatggaattaatgtctacgcgttcagtactattggctacctcagctataccctgggacaggtgttccatttctgtatcttcgggaacaggcttattgaagagagttcctcagtgatggaagccgcctactcctgtcagtggtacgacggctccgaggaagcaaagaccttcgtccagatcgtgtgccagcaatgccagaaagccatgagcatctccggggccaagttcttcacggtgtcacttgatctttttgcttcggtacttggtgcagtagttacttacttcatggtgttggtacaactcaagtaa

SEQ ID NO: 8 - DNA, 1053 bp, Spodoptera littoralis
  misc_feature (1)..(1053): PBANR receptor encoding cDNA
atgacattgtcagcgcccccgatcgacgagtttgaggacccgttcgtcgtgatgaacacgacgaatgtgtcgagtcacccggccgcctatgacgagccatacacgctcgatctggtggtgccactcaccgtcacctacgtggtgatattcgtagccggcatcctggggaacaccagcacctgtgtggtcattgcgaggaatcgctctatgcacaccgctacaaacttttacctcttcagtttagcaatttcggatctcatattgttagtatgtggtctaccgtttgaggtgcacagattatggaacccggacacgtacccgttgggcgaggctcactgcatcgccattgggttagcgtcggagacttcagctaacgcgacagtgttgacaataacagcgtttacggtggagcggtacattgcgatatgtagacctttcatgtcgcacaccatgtcgaagctgtcacgagctgtgcggtttataatagctatttgggtgtttgcgttgtgtactgcggtaccacaagcgatgcagttcggtatagtgtcgtatgtggataatggtcagaatgtgagcgcgtgcacagtgaagggtgtgggtgttcatcaagtgttcgttatatctagtttcgtgttcttcgtggtgcctatgtcgatgatatcagtgttgtacgcgttgattggtattaagttgcggatctctcgagtgttgcatccagtgaagaagctctcggtggacagcaacgaaagagcgagtggacagatgcagtacaggaatggagcatcacagaggagagtcattagaatgctcgttgcggtggcgctgtcattcttcatttgctgggcgcccttccacgtgcaaaggttgctggccatctacgggaaaagtttagaacacccttctgatactttctacttggtctacatcgtgctgacgtttttgtcgggagtgctatatttcctgtcaaccgccatcaacccgttcctgtacaacatcatgtcgaataagtttagaaacgccttcaaggtgagtttttgttacattcatgattga

SEQ ID NO: 9 - DNA, 1656 bp, Meloidogyne incognita
  misc_feature (1)..(1656): Serotonin-like receptor encoding cDNA
atgttagaaaatgatttggaaaatttggacgattcttcattaattctgcatcatttgggagggtcttttgtccatgcacaaattgtagaaattgaagaaaatgattatataaacagcaacacaagtgtaagcaatacaaatttaaatttattttcatcatcatcatcaacgtcttcctcgtcattattttcttcgtcttcacaaagatctcctcttcttgaaccaaattttgttgatgaaatttggccagctacaagttcaaattctttattttattctcatccaataattgcaattttaatctcaattttaatatttttattaattttaattactataattggaaatttgggtgtttgtgctgcaattttgttagttagaaaattaaaagcgcaaccagctaatttgttgcttatatcacttgctttggctgatttttgtgtaggcctttttgtaatgcctttagctgcagtttacgttttggaagatcgttggatatttggtgatattctctgccgtttttgggttactgccgatttaactttgtgtacagcttcaattttaaatttgtgtgcaatttctgttgatcgacatttggcagtgactagagctcttcgatattcagcattacgtactcgaagacgtatttgtttgtatatttgtattgtttggttgggtgctttattagtttctgctgcgcctttagctttgttaccgtttccaaagattgagcaatattgtcaggtctcacaaaatcgtgtttatcaaatttatgcaacaattattgcgttttggggtccatcattaataatggttattgtttatgttaaattatggagtgcagcgaaaagaatgcatcgacaagatcaattggttttgcgttggcaaggagtacatttaccttctgatggagatttagaggacggacttcctccaacaacaacaacatcaaatgctactaaatctttatttagaaatgattcaatagcccgtgcaaatgctcgttttctttttgcattgcgtatgtcagcacaaaaatttggttattatgatcatcaaaataataaaactaatggaggagaaattataaatgatgatgaaaatttaagaattgaagttagaacaagtgctagtagtagtggtggtggaggaggaggatcttctactttggggactttacgtgttccaatagtgttaattattatgagtgccttcataatttgttgggtgccattttttatattagctttggcaaaatcacagcattgggtaaattatgttcctagatggcttgattcacttacattatggcttggatattctaatagcatgttaaacccaataatttattgcaaatataatcgagaatttcgtgttccatttggagaaatgctttgttgtcggtttagaacaattcaagacgttatgagaaatgaaagttattatgcaaaattcggttctccaagaatttctgaaacaaaaattgggggaagtcaacaacggtcctctggaaataattattacactaaaagaaaaagaaacaacacagaaaggagagcaccacaaaatgggtttttagattcttcgaaaacgaagaaaaattatgggattaaaaggaatagtagtccttctatcttgatggaatcatcaaaataa

SEQ ID NO: 10 - DNA, 1601 bp, Meloidogyne incognita
  misc_feature (1)..(1601): Dopamine-like receptor encoding cDNA
atgttgccctggtggctacctcttttaatttttgtccccatcttttctgtattaatttcccttgccttttctggcaaccttctcgtaatcgcagcaatattttacgacagaaaattgcgtagacaaccagaaaatttatttttagtttccttagctttgtcagacttgtttgttgctggaatggttatggttttagcggcagcaaacgatttaattggtttttggccttttggggctgctttttgtcaattttggatttgtttggatattgcttgtagtactgcttctattttaaatttatgtgcaatagctctcgatcgttttattcacattagcaggccaatgcgttatgttagatttgttggaagaagggttatttgttgttctgtttgtgctatttggataatttctacagctgttggaacggcccaaatagcttttggggctactaatggattggagatatattctgttatatttaatggagactattttggagagattaatgagactgataaatttagattcatcgaacaacaacatacactagttcaatgccaattacaattatctcctttttatgccgttttttcttccttacttagcttcttcctaccagctactttgatgctttttctttactacaggctttatttatatgctcggcatcatgctcgttctatacaatcacaacttaaacaggctacttcacttttgatttacaattagcctcagatagagtacgtcaagtagttgtacgtccttctagtggatttttatccccctcttcaatagcccttcactacgattctcgaagatttagtggaaaatcctcctttgatggagaatcacttcaaaaagccgagtttagccaaataaacgttgctgtgaataaatcaccagaaaatcattctatcgacgctacaatccaacaaggaacgcctgtacttagggctacattgagacaattaagaaggacagaaagcaataatagtagcagctttagctttgtttcgagaaatggattgggtgttaatagcagaagggttggttgtggtggtggtaatggtggggagagaagaaagagttgttccccacttccttctccaacaacaaaaagaggttttaaatcttgtgggtggtctaataataataatggcaatggagggcatcgttcttctgacaaaaaagcacgtgttactcttggtgttattatgggtacttttctaatatgttggttacccttctttattgtcaatgttctacgccctttctggcctgaaatttttcctccactactttttcagacagtttcctggctaggttatgccaattcttcagtaaatccagtaatttatggaatatttaatcgagactttcgacgtgcctttagccgtataatgaacaaattaattcattgtatagacgaggaaagaaggggagaaaatagtcgaggagggacatttagaactagaacagaaagtgaatcttttaataaatttaataaaagtttaaataatgcaagtgaactacaacaacaacaaaaattaatttttaaacaaaaaaagacaaataaaaaattgagatttttatgtttttaa

SEQ ID NO: 11 - DNA, 1484 bp, Meloidogyne incognita
  misc_feature (1)..(1484): Neuropeptide receptor encoding cDNA
atggaagcatctacaatggaattacttccacctcccaccatattatttaatgtatcatcagcgccttctgttatttgtgaggatatgaatgcttatttatggaacactcgtcgggatttgactacatgtcctttcgttatggctctatttgcttccttaattcatcaatcattttactgggtattatcggaaactcttgtgtgattatggctattgccagaattaaatcacttcaaactgtgcctaatatgtttattttctcgttgagttgttcagatatacttgtttgctttatttctgctactataactccaatcgctgccttcaaaaaggattggatttttggccaattcttatgctcttttgcaccttttgttgcgggagggagtttgtgtttctctacattcactttggcggctatttccgtggaccgttttcttctaatactttttcccacgagaaaggctttctcgcatacacaagctttatttataattcttgtcacgtttctgttggctagcgggtttagtttaccaatgttgttcatgcaaaagttaaaaccagtaactcattattgtggccgattttgtttcgaggattggggcgagatgattggaataagacgaatctatggaactttattgctgaccgtccaattcattattccgttagcattgatcactttctgttacacagcaatttcattccgacttggaaaaagcgtgaatctacgtactaggaagaaatgtgaatggcaaatgccgattagtgctcagcgaaacgcggccacaaagcgaagacaacgaactaaccgaatgtttattgcaatggtaattgccttctcagtgagctggatatggtcggttctatataatatgttacgagactatgacggtcttccaaagtttgtacatgatcaagaattcttttttggaatattaacacactgtattgcaatgagctccacggtgtggaatccgatcttatatgccttattaaatctacaactaagggcagcctttctacaacttgtgccacaatgtattaagcattctctgacttgcacttgtagtcctaataaaggaagattaagcggtcgatcagttgctcgatgtgcctaccttgaagagagccggcttcttggttcctcagttcttcgcggaacaaccggacaagtacagagaagagaaacgaattatgaaaataaaaactgttttaattgtttgattggctcctctagatccgttgaatcttcttcaaacgaaacacagaagcaaacgttttctgttaaatgtgaaggaaataaaggagatgctaaaattttgtcacataatcaccatcggcatcaattagaaagaataacagcggacggtggatttgacgagcaaacatggacagagcaactgaggaaggactctgaagatgatgccttagatgattgtaatgatgatttgagagaaagagatacagatgagaggatatga

SEQ ID NO: 12 - DNA, 57 bp, Artificial
  AlstAR degenerate 1Forward Primer
atgacgacgtatgcacgtacagctacagctaatctctagcttctagctattcaagtc

SEQ ID NO: 13 - DNA, 51 bp, Artificial
  AlstAR degenerate 2Forward primer
gtagctccagcttttcacagctgcagctacagctgatctatcgtagctatg

SEQ ID NO: 14 - DNA, 52 bp, Artificial
  AlstAR degenerate 1Reverse primer
gtagctacagctacgagctatggtagctgtagctgtagctgtagctgtagct

SEQ ID NO: 15 - DNA, 51 bp, Artificial
  AlstAR degenerate 2Reverse primer
tggatsagcttgtcgtagctaatcccatcggtatgcacttctatcgcagct

SEQ ID NO: 16 - DNA, 20 bp, Artificial
  3′RACE amplification oligonucleotide 1s
gcatcccatagcttccatgt

SEQ ID NO: 17 - DNA, 31 bp, Artificial
  3′RACE amplification oligonucleotide 2s
ggtgatattaaccacagctattcccgtgggc

SEQ ID NO: 18 - DNA, 31 bp, Artificial
  3′RACE amplification oligonucleotide 3s
gtatgctgacgaggttgtggaagagtgctcc

SEQ ID NO: 19 - DNA, 21 bp, Artificial
  3′RACE amplification oligonucleotide 4s
aaggtgacgagaatggttgtg

SEQ ID NO: 20 - DNA, 21 bp, Artificial
  3′RACE amplification oligonucleotide 5s
gcgcagatagtgtcgcatgta

SEQ ID NO: 21 - DNA, 31 bp, Artificial
  5′RACE amplification oligonucleotide 1as
agacgactcttccacaacctcgtcagcatac

SEQ ID NO: 22 - DNA, 20 bp, Artificial
  5′RACE amplification oligonucleotide 2as
gttaatatcaccacccatat

SEQ ID NO: 23 - DNA, 31 bp, Artificial
  5′RACE amplification oligonucleotide 3as
gcccacgggaatagctgtggttaatatcacc

SEQ ID NO: 24 - DNA, 22 bp, Artificial
  oligo SL1
ggtttaattacccaactttgag

SEQ ID NO: 25 - DNA, 41 bp, Artificial
  oligo(dT) SL1
ggtttaattacccaactttgagttttttttttttttttacg

SEQ ID NO: 26 - DNA, 18 bp, Artificial
  AlstAR gene amplification primer
atggcgtcgactgaagac

SEQ ID NO: 27 - DNA, 18 bp, Artificial
  AlstAR amplification primer
tcagacgatgtcatggca

SEQ ID NO: 28 - DNA, 22 bp, Artificial
  Or83b Forward primer
atgaccaaaggtgaaggcccag

SEQ ID NO: 29 - DNA, 21 bp, Artificial
  Or83b Reverse primer
gtgttggtacaactcaagtaa

SEQ ID NO: 30 - DNA, 46 bp, Artificial
  DHR Forward primer
tttctctatcgtatctttcaaaggaatcgtctatgacgatcgtgtc

SEQ ID NO: 31 - DNA, 44 bp, Artificial
  DHR Reverse primer
aagtcttatcggtatcgatatcgaagatcgacccacatatcgat

SEQ ID NO: 32 - DNA, 27 bp, Artificial
  3′RACE DHR amplification oligonucleotide 1s
aacctcatgtcgacgtatattctgtct

SEQ ID NO: 33 - DNA, 27 bp, Artificial
  3′RACE DHR amplification oligonucleotide 2s
atgcttgtagaaggtttgtacctgtac

SEQ ID NO: 34 - DNA, 27 bp, Artificial
  3′RACE DHR amplification oligonucleotide 3s
tgggttatatgcaggtgcttcgtcaac

SEQ ID NO: 35 - DNA, 27 bp, Artificial
  5′RACE DHR amplification oligonucleotide 1as
ggatggggtgcgccggcggtgttccta

SEQ ID NO: 36 - DNA, 27 bp, Artificial
  5′RACE DHR amplification primer 2as
catacatatgaccagaatcgtacacga

SEQ ID NO: 37 - DNA, 27 bp, Artificial
  5′RACE DHR amplification oligonucleotide 3as
ggcgaggtagatgaggctggtgacgtc

SEQ ID NO: 38 - DNA, 21 bp, Artificial
  DHR Forward amplification primer
atggcggagaagtgcctggcg

SEQ ID NO: 39 - DNA, 21 bp, Artificial
  DHR Reverse amplification primer
tcataccgtgagtcgtatgct

SEQ ID NO: 40 - DNA, 53 bp, Artificial
  LGR1 degenerate forward primer
gcatgctatctctagctacagctcatcgcagctagcttttccatctgtctgtc

SEQ ID NO: 41 - DNA, 46 bp, Artificial
  LGR1 degenerate reverse primer
tcagctatcagctggagctaagagcaagctatagctgcatagctgt

SEQ ID NO: 42 - DNA, 18 bp, Artificial
  3′RACE LGR1 amplification oligonucleotide 1s
tgatgaaaatgccttcgc

SEQ ID NO: 43 - DNA, 20 bp, Artificial
  3′RACE LGR1 amplification oligonucleotide 2s
cacgaaccagtcaacaacac

SEQ ID NO: 44 - DNA, 27 bp, Artificial
  3′RACE LGR1 amplification oligonucleotide 3s
gaaagcgtacctgacgcatcatttcca

SEQ ID NO: 45 - DNA, 27 bp, Artificial
  3′RACE LGR1 amplification oligonucleotide 4s
gttatcagtaattactttaactatagt

SEQ ID NO: 46 - DNA, 21 bp, Artificial
  5′RACE LGR1 amplification oligonucleotide 1as
actgccggagtctgagaagta

SEQ ID NO: 47 - DNA, 20 bp, Artificial
  5′RACE LGR1 amplification oligonucleotide 2as
gtactcctcgctcgtagtcg

SEQ ID NO: 48 - DNA, 20 bp, Artificial
  5′RACE LGR1 amplification oligonucleotide 3as
catttcgactgcattatagc

SEQ ID NO: 49 - DNA, 21 bp, Artificial
  5′RACE LGR1 amplification oligonucleotide 4as
gccttctaggtctatagcttg

SEQ ID NO: 50 - DNA, 27 bp, Artificial
  LGR1 amplification forward primer
atgtattggagattatgtatttgggct

SEQ ID NO: 51 - DNA, 27 bp, Artificial
  LGR1 amplification reverse primer
ttaaagcggtacctcactactgtcttt

SEQ ID NO: 52 - DNA, 26 bp, Artificial
  Oct/TyrR degenerate forward primer
ccagaatgggaaggcatattctgcac

SEQ ID NO: 53 - DNA, 27 bp, Artificial
  Oct/TyrR degenerate reverse primer
acagcccattattgatagcctcagagg

SEQ ID NO: 54 - DNA, 18 bp, Artificial
  3′RACE Oct/TyrR amplification oligonucleotide 1s
ccagaaaattgacaccaa

SEQ ID NO: 55 - DNA, 20 bp, Artificial
  3′RACE Oct/TyrR amplification oligonucleotide 2s
gagagtaactcgaaagaaac

SEQ ID NO: 56 - DNA, 24 bp, Artificial
  3′RACE Oct/TyrR amplification oligonucleotide 3s
tgctgtttatcaattcattgaaga

SEQ ID NO: 57 - DNA, 22 bp, Artificial
  5′RACE Oct/TyrR amplification oligonucleotide 1as
agttctttttttagtggccaaa

SEQ ID NO: 58 - DNA, 19 bp, Artificial
  5′RACE Oct/TyrR amplification oligonucleotide 2as
gacaaggcgtatcaggttc

SEQ ID NO: 59 - DNA, 26 bp, Artificial
  5′RACE Oct/TyrR amplification oligonucleotide 3as
accctagaagtggcggagagctaata

SEQ ID NO: 60 - DNA, 21 bp, Artificial
  Oct/TyrR amplification forward primer
atggggcaaacagctacacac

SEQ ID NO: 61 - DNA, 18 bp, Artificial
  Oct/TyrR amplification reverse primer
ctctgtatgaaaccgtga

SEQ ID NO: 62 - DNA, 24 bp, Artificial
  PBANR amplification forward primer
atgacattgtcagcgcccccgatc

SEQ ID NO: 63 - DNA, 18 bp, Artificial
  PBANR amplification reverse primer
tcaatcatgaatgtaaca

SEQ ID NO: 64 - DNA, 30 bp, Artificial
  AlstCR degenerate forward primer
gaagtgtctttctctagctatatcggatgc

SEQ ID NO: 65 - DNA, 30 bp, Artificial
  AlstCR degenerate reverse primer
atgcgcagcaatgcgtagcaatgcgctctt

SEQ ID NO: 66 - DNA, 24 bp, Artificial
  AKHR degenerate forward primer
gacctgatgtgcacgacagtcatg

SEQ ID NO: 67 - DNA, 24 bp, Artificial
  AKHR degenerate reverse primer
gtcagatccaagtaccacagagca

SEQ ID NO: 68 - DNA, 26 bp, Artificial
  Forward primer for cloning AlstAR
ggaattccatggcgtcgactgaagac

SEQ ID NO: 69 - DNA, 26 bp, Artificial
  Reverse primer for cloning AlstAR
cctcgaggtcagacgatgtcatggca

SEQ ID NO: 70 - DNA, 28 bp, Artificial
  Forward primer for cloning AlstCR
ggaattccctgcgttccattcactgcta

SEQ ID NO: 71 - DNA, 28 bp, Artificial
  Reverse primer for cloning AlstCR
cctcgaggaagaattgggttcatggcag

SEQ ID NO: 72 - DNA, 29 bp, Artificial
  Forward primer for cloning DHR
ggaattccgatggcggagaagtgcctggc

SEQ ID NO: 73 - DNA, 29 bp, Artificial
  Reverse primer for cloning DHR
cctcgagggtcataccgtgagtcgtatgc

SEQ ID NO: 74 - DNA, 30 bp, Artificial
  Forward primer for cloning AKH
ggaattccggacctgatgtgccgagtcatg

SEQ ID NO: 75 - DNA, 28 bp, Artificial
  Reverse primer for cloning AKH
cctcgagggcttgtcgatccaataccac

SEQ ID NO: 76 - DNA, 37 bp, Artificial
  Forward primer for cloning LGR1
cgggatcccggatgtattggagattatgtatttgggc

SEQ ID NO: 77 - DNA, 29 bp, Artificial
  Reverse primer for cloning LGR1
cctcgagggttaaagcggtacctcactac

SEQ ID NO: 78 - DNA, 31 bp, Artificial
  Forward primer for cloning Or83b
ggaattccgatgaccaaagtgaaggcccagg

SEQ ID NO: 79 - DNA, 31 bp, Artificial
  Reverse primer for cloning Or83b
cctcgagggttacttgagttgtaccaacacc

SEQ ID NO: 80 - DNA, 28 bp, Artificial
  Forward primer for cloning Oct/TyrR
ggaattccggggcaaacagctacacacg

SEQ ID NO: 81 - DNA, 31 bp, Artificial
  Reverse primer for cloning Oct/TyrR
cctcgagggtcacggtttcatacagagtaac

SEQ ID NO: 82 - DNA, 30 bp, Artificial
  Forward primer for cloning PBAN-R
ggaattccgatgacattgtcagcgcccccg

SEQ ID NO: 83 - DNA, 34 bp, Artificial
  Reverse primer for cloning PBAN-R
taaagcggccgctcaatcatgaatgtaacaaaaa

SEQ ID NO: 84 - DNA, 21 bp, Artificial
  MiserR Forward primer 1
gatttggaaaatttggacgat

SEQ ID NO: 85 - DNA, 21 bp, Artificial
  MiserR Forward primer 2
ggagggtcttttgtccatgca

SEQ ID NO: 86 - DNA, 21 bp, Artificial
  MiserR Forward primer 3
tctcatccaataattgcaatt

SEQ ID NO: 87 - DNA, 21 bp, Artificial
  MiserR Reverse primer 1
acgtaatgctgaatatcgaag

SEQ ID NO: 88 - DNA, 21 bp, Artificial
  MiserR Reverse primer 2
caaatttaaaattgaagcagt

SEQ ID NO: 89 - DNA, 21 bp, Artificial
  MiserR Reverse primer 3
agccaaagcaagtgatataag

SEQ ID NO: 90 - DNA, 21 bp, Artificial
  MidopR Forward primer 1
actcttggtgttattatgggc

SEQ ID NO: 91 - DNA, 21 bp, Artificial
  MidopR Forward primer 2
tggctaggttatgccaattct

SEQ ID NO: 92 - DNA, 21 bp, Artificial
  MidopR Forward primer 3
cgagactttcgacgtgccttt

SEQ ID NO: 93 - DNA, 21 bp, Artificial
  MidopR Reverse primer 1
aaaggcacgtcgaaagtctcg

SEQ ID NO: 94 - DNA, 21 bp, Artificial
  MidopR Reverse primer 2
agaattggcataacctagcca

SEQ ID NO: 95 - DNA, 21 bp, Artificial
  MidopR Reverse primer 3
gcccataataacaccaagagt

SEQ ID NO: 96 - DNA, 23 bp, Artificial
  MiserR Forward primer 1
atgttagaaaatgatttggaaaa

SEQ ID NO: 97 - DNA, 20 bp, Artificial
  MiserR Reverse primer 1
ttattttgatgattccatca

SEQ ID NO: 98 - DNA, 23 bp, Artificial
  MiDopR forward primer
atgttgccctggtggctacctct

SEQ ID NO: 99 - DNA, 20 bp, Artificial
  MiDopR reverse primer
ttaaaaacataaaaatctca

SEQ ID NO: 100 - DNA, 23 bp, Artificial
  MiNprR forward primer
atggaagcatctacaatggaatt

SEQ ID NO: 101 - DNA, 20 bp, Artificial
  MiNprR reverse primer
tcatatcctctcatctgtat